(19) United States
(12) Patent Application Publication
(10) Pub. No.: US 2006/0287520 A1
(43) Pub. Date: Dec. 21, 2006

(54) Synthesis of salinosporamide A and analogues thereof

(76) Inventors: Samuel J. Danishefsky, Englewood, NJ (US); Atsushi Endo, Boston, MA (US)

Correspondence Address: CHOATE, HALL & STEWART LLP;SLOAN-KETTERING INSTITUTE FOR CANCER RESEARCH, TWO INTERNATIONAL PLACE, BOSTON, MA, 02110, US

(21) Appl. No.: 11/434,698

(22) Filed: May 16, 2006

Related U.S. Application Data
(60) Provisional application No. 60/681,454, filed on May 16, 2005.

(51) Int. Cl. C07D 487/02

(52) U.S. Cl. 540/203; 548/453

(57) ABSTRACT

A novel synthesis of salinosporamide A is provided. Salinospoamide A as well as structurally related natural products, omuralide and lactacystin, have been shown to be proteasome inhibitors. Therefore, these compounds as well as analogues of these natural products may be useful in the treatment of proliferative diseases such as cancer, autoimmune diseases, diabetic retinopathy, etc. The invention provides for the synthesis of salinosporamide A as well as analogs thereof using a convenient point for derivatization of the bicyclic core. Pharmaceutical compositions and method of using the inventive compounds are also provided.

### RELATED APPLICATIONS

The present application claims priority under 35 U.S.C. § 119(e) to U.S. provisional patent application, U.S. Ser. No. 60/681,454, filed May 16, 2005, which is incorporated herein by reference.

### GOVERNMENT SUPPORT

The work described herein was supported, in part, by grants from the National Institutes of Health (CA-103823; NCI Core Grant No. 08748). The United States government may have certain rights in the invention.

### BACKGROUND OF THE INVENTION

The proteasome-mediated pathway in cellular protein degradation serves as a crucial regulatory step for many cellular processes including cell proliferation and apoptosis. Therefore, proteasome inhibitors have been aggressively investigated in the search for a novel class of anticancer drugs (Adams, Nat. Rev. Cancer 4:349, 2004; incorporated herein by reference). Salinosporamide A (1), isolated from a marine actinomycetes bacteria by Fenical and co-workers at the Scripps Institute of Oceanography (Feling et al. Angew. Chem. Int. Ed. 42:355, 2003; incorporated herein by reference), is a highly cytotoxic proteasome inhibitor, and is structurally related to omuralide (2) and lactacystin (3) (Omura et al. J. Antibiot. 44:113, 1991; Omura et al. J. Antibiot. 44:117, 1991; Corey et al. J. Am. Chem. Soc. 114:10677, 1992; Fenteany et al. Proc. Natl. Acad. Sci. U.S.A. 91:3358, 1994; Fenteany et al. Science 268:726, 1995; Fenteany et al. J. Biol. Chem. 273:8545, 1998; Corey et al. Chem. Pharm. Bull. 47:1, 1999; Masse et al. Eur. J. Org. Chem. 2513, 2000; Tomoda et al. Yakugaku Zasshi 120:935, 2000; each of which is incorporated herein by reference), themselves known as proteasome inhibitors. Salinosporamide A displays remarkable in vitro cytotoxicity (IC₅₀ of approximately 10 nM), and its activity appears to be directed to the inhibition of the 20S proteasome. Thus, salinosporamide A is approximately 35 times more potent than omuralide (2), which has the same molecular target. 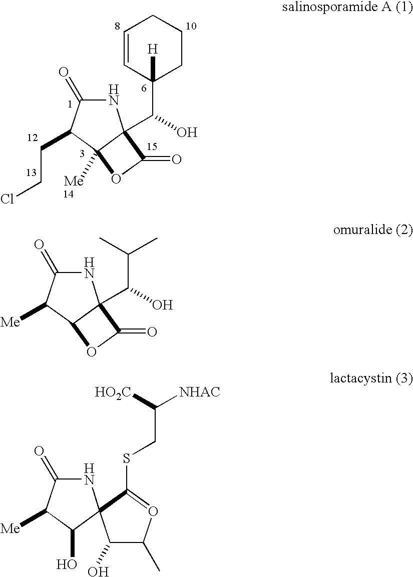
Synthetic efforts towards omuralide and lactacystin have been executed by Corey and co-workers. The first total synthesis of salinosporamide A was reported by Corey and co-workers in 2004 (Reddy et al. J. Am. Chem. Soc. 126:6230, 2004; incorporated herein by reference). Salinosporamide A is highly potent as a proteasome inhibitor and is structurally unique among proteasome inhibitors. In recent years, the search for proteasome inhibitors as anti-proliferative agents has increased. Therefore, an efficient synthesis of salinosporamide A and analogues thereof that would allow for readily preparing analogues would be useful in the search for new clinical candidates based on the core structure of salinosporamide A and omuralide.

### SUMMARY OF THE INVENTION

The present invention provides a new synthesis of salinosporamide A and analogues thereof. The synthesis is particularly useful in preparing analogues of salinosporamide A, specifically analogues with different substituents at C-2, C-4, and/or the nitrogen of the lactam ring. The compounds of the invention may be used as proteasome inhibitors. In particular, the compounds may be used to treat anti-proliferative diseases such as cancer, benign neoplasms, autoimmune diseases, and diabetic retinopathy. These compounds may be used as pharmaceutical agents themselves or may be used as lead compounds in developing new pharmaceutical agents. Pharmaceutical compositions and methods of using these compounds to treat diseases such as cancer, autoimmune diseases, diabetic retinopathy, etc. are also provided. The present invention also includes intermediates and synthetic methods useful in the preparation of salinosporamide A and analogues thereof.

In one aspect of the invention, the compounds of the invention are of the formula: 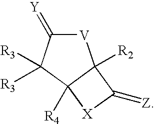
In certain embodiments, the stereochemistry is defined as shown in the formula: 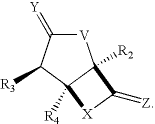
In certain particular embodiments, X, Y, Z, V, and R₄ are defined as shown in the formula: 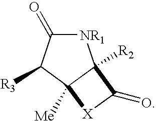
In certain embodiments, salinosporamide A and analogues thereof have anti-proliferative activity and are useful in treating diseases such as cancer, autoimmune disease, neoplasms, etc. The compounds may be proteasome inhibitors. In particular, the compounds may inhibit the 20S proteasome. In certain embodiments, the compounds are cytotoxic. The analogues of salinosporamide A provided by the invention may be more potent and/or exhibit less side effects than natural products such salinosporamide A or omuralide.

The invention also provides pharmaceutical compositions of these compounds for use in treating human diseases and veterinary diseases. The compounds of the invention are combined with a pharmaceutical excipient to form a pharmaceutical composition for administration to a subject. Methods of treating a disease such as cancer are also provided wherein a therapeutically effective amount of an inventive compound is administered to a subject.

In another aspect, synthetic methods and intermediates useful in preparing salinosporamide A or analogues thereof are provided. Such methods provide the ability to make various substitutions at R₁, R₂, and R₃ as shown in the formulae above. Other steps in the synthesis of the inventive compounds are also included within the invention.

Therefore, the novel synthesis of salinosporamide A provides methods for the preparation of the inventive compounds which may be useful in treating diseases which involve the degradation of proteins via proteasome-mediated pathways. For example, these compounds may be used in treating proliferative diseases such as cancer and autoimmune diseases. The use of the compounds in pharmaceutical compositions and treatment regimens are also provided.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex mixtures of isomers.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomer or diastereomer. Alternatively, where the molecule contains a basic functional group, such as an amino group, or an acidic functional group, such as a carboxylic acid group, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. In preferred embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxylprotecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkoxy”, or “thioalkyl” as used herein refers to an alkyl group, as previously defined, attached through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limted to, aziridinyl, pyrrolidinyl, piperidinyl, morpholinyl, pyrrolyl, imidazolyl, 1,3,4-trianolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or hetercyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rx; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Carbocycle”: The term “carbocycle”, as used herein, refers to an aromatic or non-aromatic ring in which each atom of the ring is a carbon atom.

“Independently selected”: The term “independently selected” is used herein to indicate that the R groups can be identical or different.

“Labeled”: As used herein, the term “labeled” means that a compound comprises at least one element, isotope, or chemical compound to enable the detection of the compound by any technique that would enable detection. Labels may be: a) isotopic labels, which may be radioactive or heavy isotopes, including, but not limited to, ²H, ³H, ¹³C, ¹⁴C, ¹⁵N, ³¹P, ³²P, ³⁵S, ⁶⁷Ga, ^{99m}Tc (Tc-99m), ¹¹¹In, ¹²³I, ¹²⁵I, ¹⁶⁹Yb, and ¹⁸⁶Re; b) immune labels, which may be antibodies or antigens, which may be bound to enzymes (such as horseradish peroxidase) that produce detectable agents; or c) colored, luminescent, phosphorescent, or fluorescent dyes. It will be appreciated that the labels incorporated into the compound at any position that does not substantially interfere with the biological activity or characteristic of the compound that is being detected. In certain embodiments, hydrogen atoms in the compound are replaced with deuterium atoms (²H) to slow the degradation of compound in vivo. Due to isotope effects, enzymatic degradation of the deuterated compounds may be slowed thereby increasing the half-life of the compound in vivo. In other embodiments such as in the identification of the biological target of a natural product or derivative thereof, the compound is labeled with a radioactive isotope, preferably an isotope which emits detectable particles, such as β particles. In certain other embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam), the entire contents of which are hereby incorporated by reference. In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

“Tautomers”: As used herein, the term “tautomers” are particular isomers of a compound in which a hydrogen and double bond have changed position with respect to the other atoms of the molecule. Tautomers are interconnected through a mechanism for interconversion. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, and pyridione-hydroxypyridine forms.

Definitions of non-chemical terms used throughout the specification include:

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

“Effective amount”: In general, the “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient. For example, the effective amount of a compound with anti-proliferative activity is the amount that results in a sufficient concentration at the site of the tumor to kill or inhibit the growth of tumor cells.

A “protein” or “peptide” comprises a polymer of amino acid residues linked together by peptide bonds. The term, as used herein, refers to proteins, polypeptides, and peptide of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these.

### BRIEF DESCRIPTION OF THE DRAWING

FIG. 1 shows exemplary synthetic routes to salinosporamide A analogues.

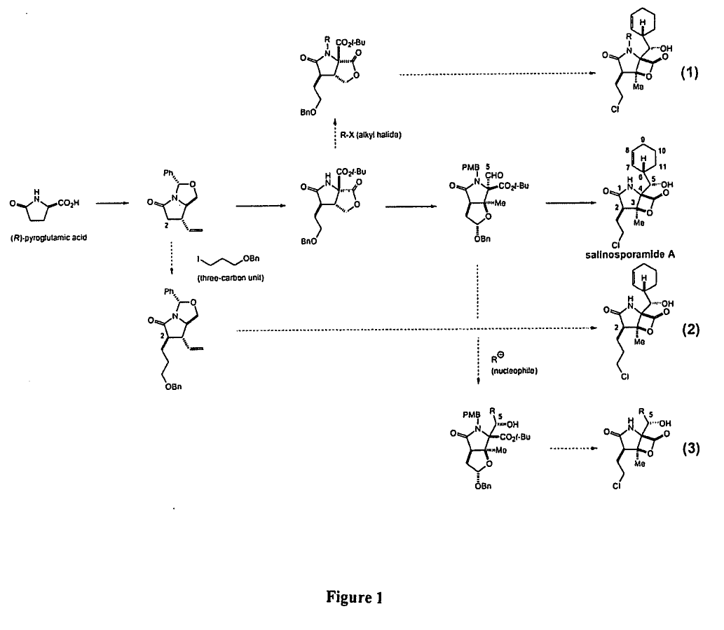

FIG. 2 shows an exemplary synthesis of various analogues of salinosporamide A from a bicyclic intermediate.

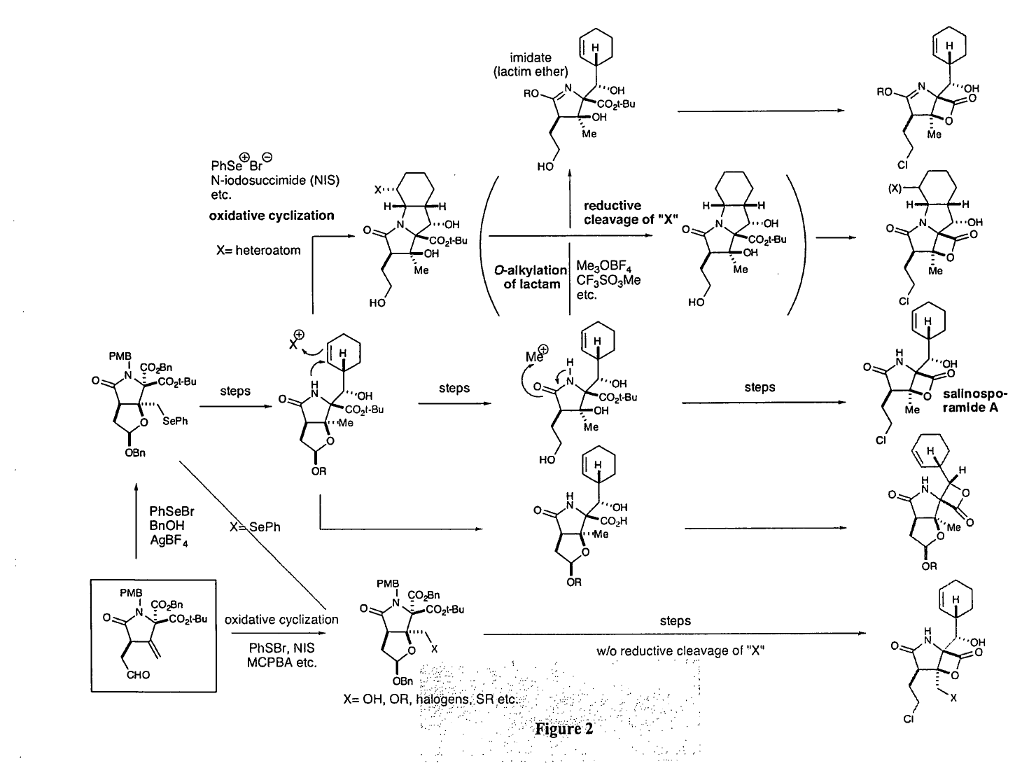

### DETAILED DESCRIPTION OF CERTAIN PREFERRED EMBODIMENTS OF THE INVENTION

The synthesis of salinosporamide A and analogues thereof is provided herein. The compounds accessible by this novel route may be proteasome inhibitors useful in the treatment of proliferative disorders such as cancer.

Compounds

In one aspect, the present invention provides compounds of the formula: 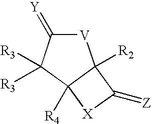
wherein

Z is O, S, C(R₅), or NR₅, wherein each occurrence of R₅ is independently hydrogen or lower alkyl;

Y is O, S, C(R₆), or NR₆, wherein each occurrence of R₆ is independently hydrogen or lower alkyl;

X is O, S, C(R₇)₂, or NR₇, wherein each occurrence of R₇ is independently hydrogen or lower alkyl;

V is O, S, C(R₁)₂, or NR₁, wherein each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, R₁, R₂, R₃, and R₄ may be taken together to form a cyclic or heterocyclic structure. In certain embodiments, a cyclic structure is formed between R₁ and R₂. In other embodiments, a cyclic structure is formed between R₂ and R₄. In yet other embodiments, a cyclic structure is formed between R₂ and R₃. In still other embodiments, a cyclic structure is formed between R₃ and R₄. In certain embodiments, a cyclic structure is formed between R₁ and R₃. In other embodiments, a cyclic structure is formed between R₁ and R₄. The cyclic structure may contain any number of atoms, either carbon atoms or heteroatoms (e.g., N, S, or O). In certain embodiments, the cyclic structure is 5- or 6-membered. In certain embodiments, both occurrences of R₃ may be taken together to form a cyclic or heterocyclic structure; ═O; ═C(R_{C})₂; ═CHR_{C}; ═NH; or ═NR_{C}.

In certain embodiments, the stereochemistry of the core bicyclic ring system is defined as shown in the formula:

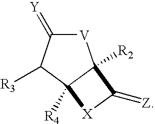

In other embodiments, the stereochemisty of the compound is further defined as shown in the formula:

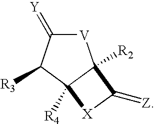

In certain embodiments, Z is O. In other embodiments, Z is S. In yet other embodiments Z is NR₅, wherein R₅ is hydrogen or lower alkyl, preferably hydrogen.

In certain embodiments, Y is O. In other embodiments, Y is S. In yet other embodiments, Y is NR₆, wherein R₆ is hydrogen or lower alkyl, preferably hydrogen.

In certain embodiments, X is O. In other embodiments, X is NH. In yet other embodiments, X is NR₇, wherein R₇ is lower alkyl.

In certain embodiments, V is O. In other embodiments, V is NH. In yet other embodiments, X is NR₁, wherein R₁ is defined as above. In certain embodiments, R₁ is cyclic or acyclic, substituted or unsubstituted, branched or unbranched aliphatic. In certain embodiments, R₁ is a substituted or unsubstituted, branched or unbranched alkyl group, preferably C₁-C₁₂ alkyl, more preferably C₁ to C₆ alkyl (e.g., methyl, hydroxymethyl, ethyl, n-propyl, i-propyl, cyclopropyl, n-butyl, i-butyl, t-butyl, pentyl, cyclopentyl, cyclohexyl, or hexyl). In certain embodiments, R₁ is an alkyl group with greater than 6 carbon atoms. In certain embodiments, R₁ is alkenyl or alkynyl. In certain embodiments, when V is —NR₁—, R₁ is not hydrogen. In certain embodiments, when V is —NR₁—, R₁ is not hydrogen or C₁-C₆ alkyl. In certain embodiments, V is not O. In other embodiments, V is not S. In certain embodiment, V is not O, NR₁, or S, wherein R₁ is H or C₁-C₆ alkyl. In certain embodiments, when Y is O, X is O, Z is O, R₄ is methyl, R₂ is 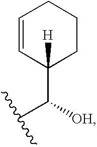
and R₃ is —CH₂CH₂Cl, then V is not NH. In certain embodiments, when Y is O, X is O, Z is O, R₄ is methyl, R₂ is 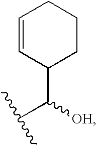
and R₃ is —CH₂CH₂Cl, then V is not NH. In other embodiments, R₁ is cyclic or acyclic, substituted or unsubstituted, branched or unbranched heteroaliphatic. In yet other embodiments, R₁ is substituted or unsubstituted, branched or unbranched acyl. In certain embodiments, R₁ is acetyl. In still further embodiments, R₁ is substituted or unstubstituted, branched or unbranched aryl or heteroaryl. R₁ may be a five- or six-membered aryl or heteroaryl group. In certain embodiments, R₁ is a substituted or unsubstituted phenyl group. In certain other embodiments, R₁ is an amide nitrogen protecting group (e.g., allyl group, 1-butyl group, Dcpm (dicyclopropylmethyl), MOM (methoxymethyl), MTM (methylthiomethyl), BOM (benzyloxymethyl), PMB (para-methoxybenzyl), trichloroethoxymethyl, t-butyldimethylsiloxymethyl, pivaloyloxymethyl, cyanomethyl, pyrrolidinomethyl, methoxy, benzyloxy, methylthio, triphenylmethylthio, TBDMS (t-butyldimethylsilyl), TIPS (triisopropylsilyl), 4-methoxyphenyl, 4-(methoxymethoxyphenyl), 2-methoxy-1-napthyl, benzyl, 4-methoxybenzyl, 2,4-dimethoxybenzyl, 3,4-dimethoxybenzyl, 2-acetoxy-4-methoxybenzyl, o-nitrobenzyl, DAM (dianisymethyl), DMTr (bis(4-methoxyphenyl)phenylmethyl, bis(4-methylsulfinylphenyl)methyl, triphenylmethyl, 9-phenylfluorenyl (Pf), bis(trimethylsilyl)methyl, t-butoxycarbonyl, benzyloxycarbonyl, methoxycarbonyl, ethoxycarbonyl, p-toluenesulfonyl, butenyl, (E)-2-(methoxycarbonyl)vinyl), DEM (diethoxymethyl), 1-methoxy-2,2-dimethylpropyl, 2-(4-methylphenylsulfonyl)ethyl).

In certain embodiments, X is O; Z is O; Y is O; and V is NR₁. In other embodiments, X is O; Z is O; Y is O; and V is NH.

In certain embodiments, R₂ is a cyclic or acyclic, substituted or unsubstituted, branched or unbranched aliphatic or heteroaliphatic moiety. In certain embodiments, R₂ is 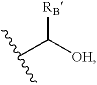
wherein R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}″; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, R₂ is 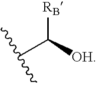
In other embodiments, R₂ is 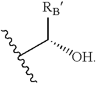
In certain embodiment, R_{B}′ is a substituted or unsubstituted, branched or unbranched, cyclic or acyclic aliphatic or heteroaliphatic moiety. In other embodiments, R_{B}′ is a substituted or unsubstituted, branched or unbranched, cyclic aliphatic or heteroaliphatic moiety. In other embodiments, R_{B}′ is a substituted or unsubstituted, branched or unbranched cyclic aliphatic moiety. In other embodiments, R_{B}′ is a substituted or unsubstituted, branched or unbranched cyclic heteroaliphatic moiety. In certain embodiments, R_{B}′ is a substituted or unsubstituted, branched or unbranched 5- or 6-membered carbocyclic ring. In other embodiments, R_{B}′ is a substituted or unsubstituted, branched or unbranched 5- or 6-membered heterocyclic ring. In certain particular embodiments, R₂ is 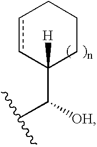
wherein n is 0, 1, 2, or 3, preferably, 1; and the dashed line represents a bond or an absence of a bond, preferably the presence of a bond. In certain embodiments, R₂ is 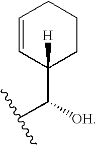
In other embodiments, R₂ is not 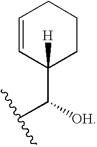
In certain embodiments, R₂ is not 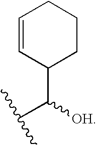
In certain embodiments, R_{B}′ is not a substituted or unsubstituted cyclohexenyl moiety. In certain embodiments, R_{B}′ is not an unsubstituted cyclohexenyl moiety. In certain embodiments, R_{B}′ is a substituted or unsubstituted, branched or unbranched aryl or heteroaryl group.

In certain embodiments, when Y is O, V is NH, X is O, Z is O, R₃ is —CH₂CH₂Cl, and R₄ is methyl, then R₂ is not 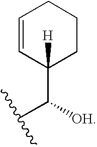
In other embodiments, when Y is O, V is NH, X is O, Z is O, R₃ is —CH₂CH₂Cl, and R₄ is methyl, then R₂ is not 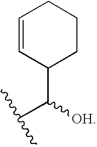
In other embodiments, when Y is O, V is NH, X is O, Z is O, R₃ is —CH₂CH₂Cl, and R₄ is methyl, then R₂ is not 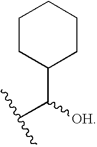
In other embodiments, when Y is O, V is NH, X is O, Z is O, R₃ is 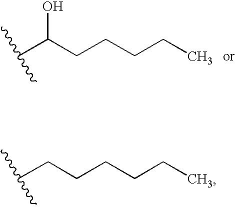
and R₄ is methyl, then R₂ is not 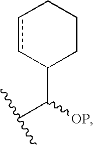
wherein the dashed line represents a bond or the absence of a bond, and P is hydrogen or acetyl, and wherein the cyclohexenyl ring is substituted with 0 to 2 hydroxyl groups.

In certain embodiments, R₃ is —CH₂CH₂X, wherein X is H, F, Cl, Br, I, —OH, or —OP, wherein P is an oxygen protecting group (e.g., silyl protecting group, Bn, alkyl, etc.). In certain embodiments, R₃ is —CH₂CH₂Cl. In other embodiments, R₃ is not —CH₂CH₂Cl. In yet other embodiments, R₃ is not —CH₂CH₂X, wherein X is H, F, Cl, Br, or I. In certain embodiments, R₃ is not 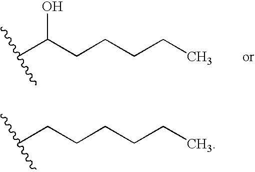
In certain embodiments, R₃ is not methyl. In certain embodiments, R₃ is cyclic or acyclic, substituted or unsubstituted, branched or unbranched aliphatic. In certain particular embodiments, R₃ is an acyclic, substituted or unsubstituted aliphatic. In yet other embodiments, R₃ is an acyclic, substituted or unsubstituted aliphatic, preferably having 1-12 carbon atoms, more preferably, having 1-6 carbon atoms. In certain embodiments, R₃ is a substituted or unsubstituted methyl, ethyl, n-propyl, or n-butyl group. In other embodiments, R₃ is cyclic or acyclic, substituted or unsubstituted, branched or unbranched heteroaliphatic. In certain embodiments, R₃ is 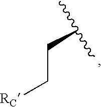
wherein R_{C}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}″; —C(═O)R_{C}″; —CO₂R_{C}″; —CN; —SCN; —SR_{C}″; —SOR_{C}″; —SO₂R_{C}″; —NO₂; —N(R_{C}″)₂; —NHC(O)R_{C}″; or —C(R_{C}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, R_{C}′ is halogen. In certain embodiments, R_{C}′ is fluorine, chlorine, bromine, or iodine, preferably chlorine or fluorine, more preferably chlorine. In other embodiments, R_{C}′ is hydroxyl, alkoxy, amino, alkylamino, dialkylamino, sulfhydryl, or acyl.

In certain embodiments, R₄ is cyclic or acyclic, substituted or unsubstituted, branched or unbranched aliphatic. In certain particular embodiments, R₄ is acyclic, unsubstituted, unbranched aliphatic. In certain embodiments, R₄ is C₁-C₆ alkyl. In certain embodiments, R₄ is methyl, ethyl, n-propyl, or iso-propyl. In certain particular embodiments, R₄ is methyl. In other embodiments, R₄ is not methyl. In certain other embodiments, R₄ is hydrogen. In still other embodiments, R₄ is not hydrogen. In certain embodiments R₄ is not hydrogen or methyl.

In certain embodiments, the compound is of the formula:

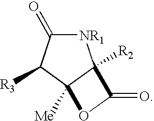

In certain embodiments, substitution about the bicyclic core structure are of the formula:

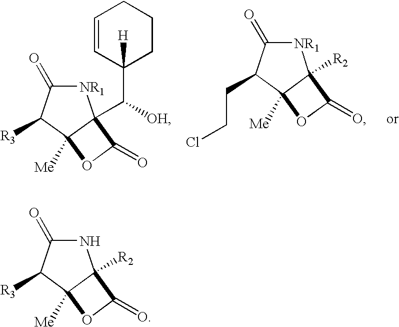

In certain embodiments, the compounds of the invention have only one change as compared to the natural product salinosporamide A, for example,

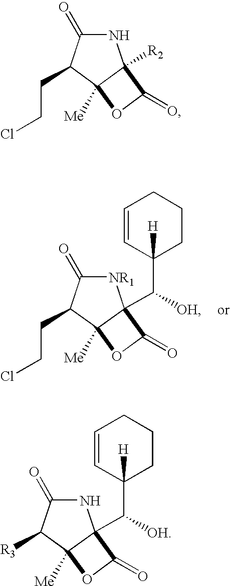

In other embodiments, the ring system of compound is altered. For example, ring systems of the following formulae are accessible by the synthetic methods described herein: 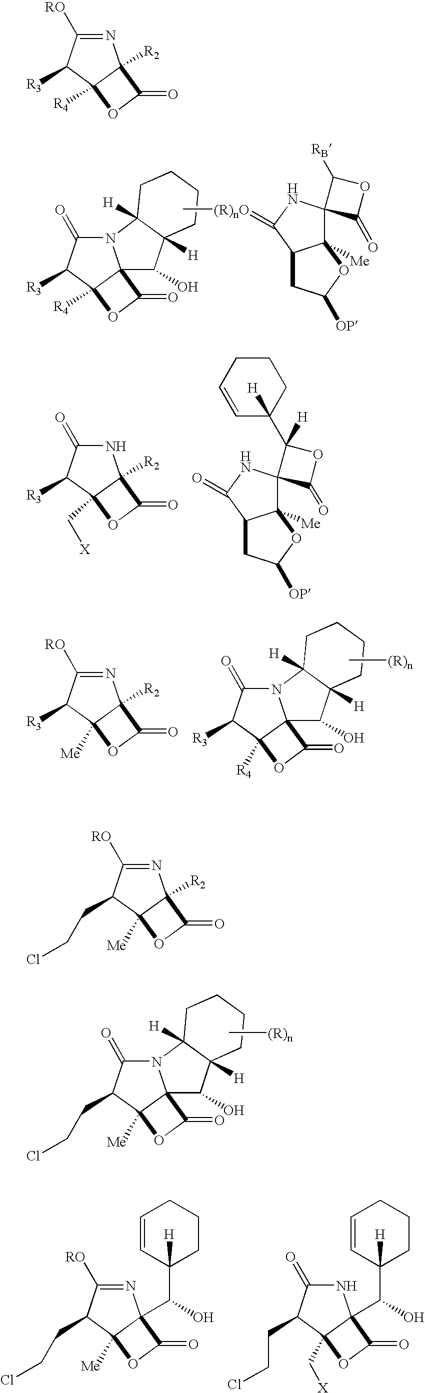
wherein each occurrence of R is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

X is hydroxyl, alkoxy, halogen, sulfhydryl, alkylthioxy, amino, alkylamino, or dialkylamino; and

n is an integer between 0 and 8, inclusive; and pharmaceutically acceptable salts thereof.

As will be appreciated by one of skill in this art, compounds of invention include derivatives, labeled forms, salts, pro-drugs, isomers, and tautomers thereof. Derivatives include protected forms. Salts include any pharmaceutically acceptable salts including HCl, HBr, HI, acetate, sulfonate (e.g., besylate, p-toluenesulfonate, mesylate, etc.) and fatty acid (e.g., lactate, citrate, myristoleate, oleate, valerate) salts.

As will be appreciated by one of skill in this art, the invention includes compositions in which the compounds are at least 90%, 95%, 98%, 99%, or 99.9% pure.

Methods of Synthesis

A novel synthesis of salinosporamide A is shown in the scheme below. As will be appreciated by one of skill in this art, various modification can be made to the starting materials and reagents used in the scheme to provide the compounds of the invention, including useful intermediates.

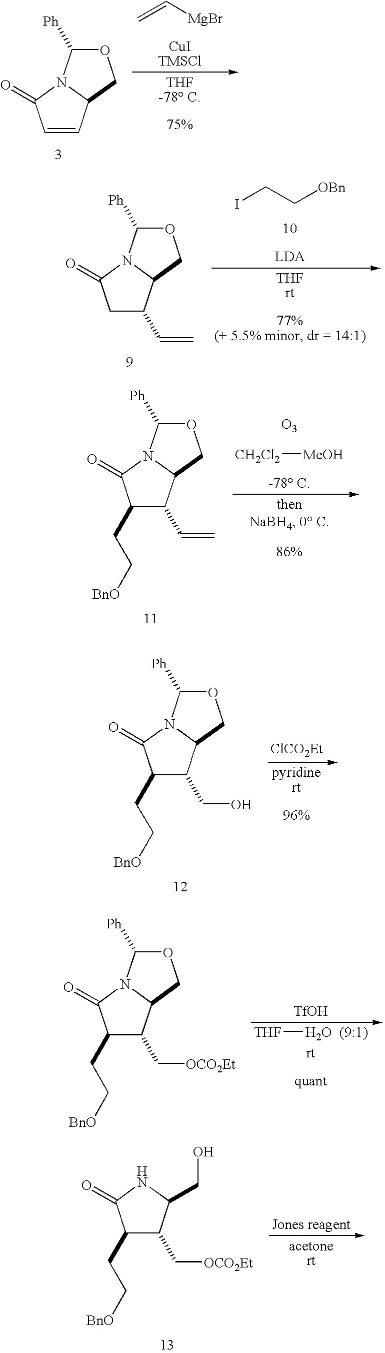

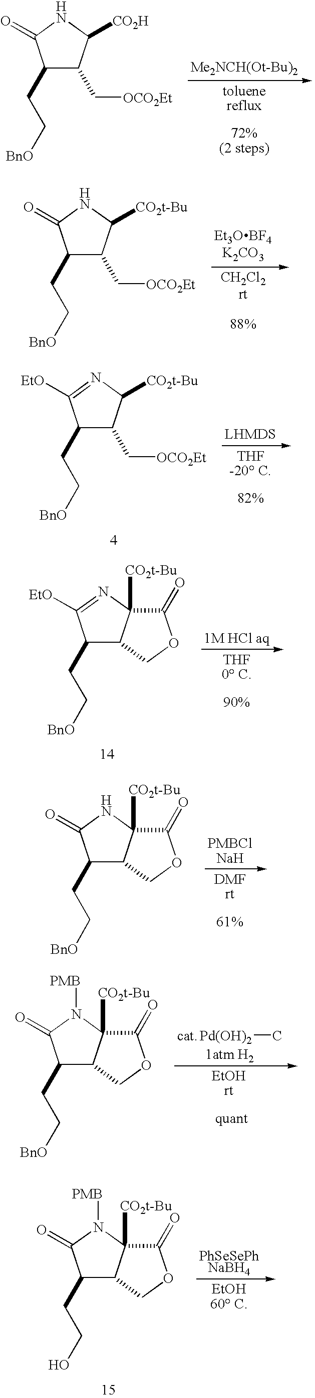

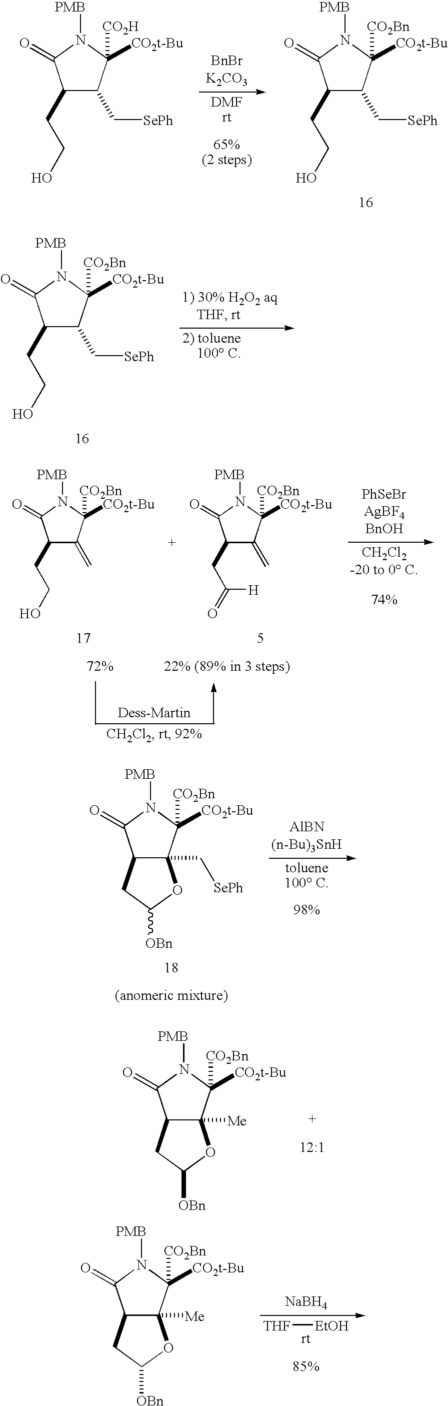

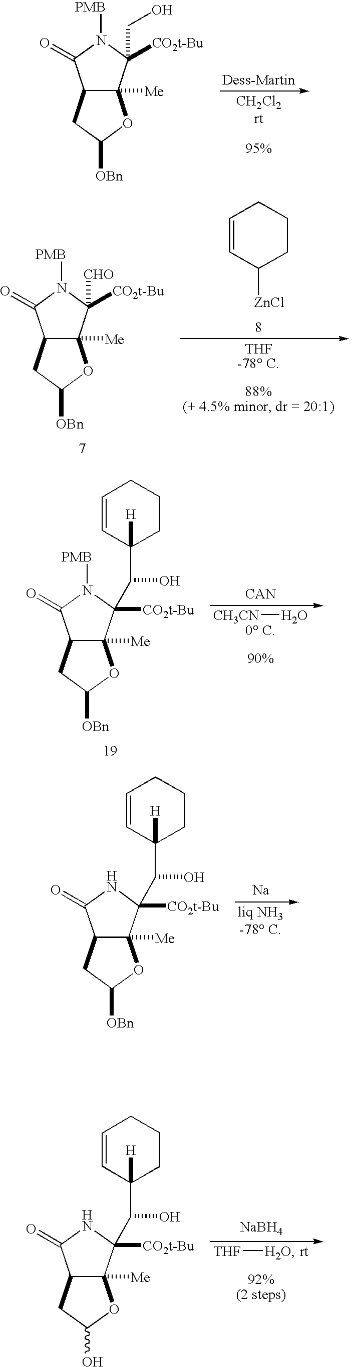

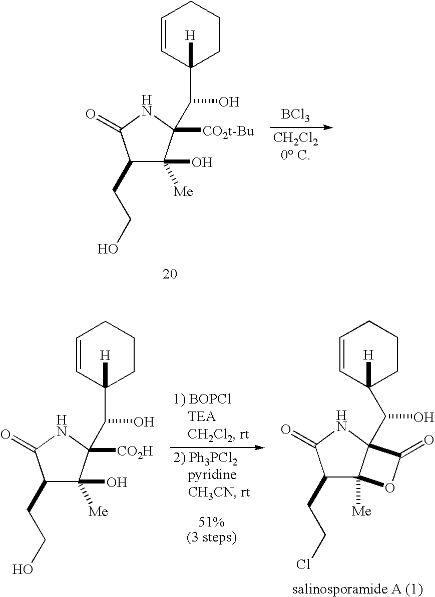

The synthesis of salinosporamide begins with the preparation of the chiral, conformationally constrained bicyclic enamide 5 from readily available (R)-pyroglutamic acid. Enamide 5 was then treated with vinyl cuprate and TMSCl, and conjugate addition proceeded from the less hindered exo-face of the bicyclic stem to afford 6 as a single product. Subsequent alkylation was effected by treatment of 6 with LDA and the iodide 7 to furnish the all-trans α,β,γ-substituted lactam 8. The stereoselection was regulated by orientation of the vinyl group on the O-position of 6. Ozonolysis of 8 followed by reductive treatment with a reducing agent such as NaBH₄ gave rise to the alcohol 9, and the corresponding ethyl carbonate was subjected to acidic cleavage of the N, O-acetal to afford 10.

The hydroxymethyl lactam 10 was converted to the imidate ester 11 by a sequence of Jones oxidation, esterification, and treatment with Meerwein reagent. The employment of an ethyl imidate served efficiently as a means not only for temporarily masking the lactam nitrogen but also for facilitating enolate formation of 11. Treatment of 11 with base (e.g., LHMDS) invoked interal acylation with the pendant ethylcarbonate to furnish the cyclic lactone 12.

Acidic treatment of 12 led to restoration of the original lactam ring, followed by N-alkylation with PMBCl, and hydrogenolysis of the benzyl ether. The lactone ring of 13 was successfully opened in an S_{N}2 fashion by treatment with in situ generated phenyl selenium anion, and the carboxylic acid moiety thus liberated was subjected to esterification to furnish 14 bearing a properly differentiated diester moiety. A sequence of selenide oxidtion followed by elimination proceeded smoothly to afford the desired ene-ol 15, unexpectedly, accompanied with the one step-advanced intermediate 16. Oxidation of purified 15 was effected by treatment with Dess-Martin periodinane to give rise to the ene-al 16.

The ene-al 16 served as a substrate for glycosyl-selenocyclization to complete stereocontrolled assembly of two consecutive quaternary centers (C3 and C4). Treatment of 16 with PhSeBr, in the presence of AgBF₄, induced (1) carbonyl-assisted phenylselenenylation of the exocyclic methylene, and (2) acetal formation with co-existing BnOH to furnish the benzyl glycoside 17. Reductive deselenenylation was conducted under standard radical conditions (e.g., AIBN, n-Bu₃SnH) to furnish the methyl group. The major isomer, separable at this stage, was subjected to further transformations. The benzyl ester was selectively reduced with a reducing agent, such as NaBH₄, and the corresponding alcohol was treated with Dess-Martin periodinane to give rise to the bicyclic aldehyde 18.

The cyclohexenyl group was installed by reacting the zinc reagent 19 with the aldehyde 18 to afford the desired adduct 20. The protecting group of 21 was cleaved by ceric ammonium nitrate (CAN)-mediated oxidation, followed by reductive opening of the benzyl glycoside by a sequence of Birch reduction and treatment with a reducing agent such as NaBH₄ to afford the triol 21. Acidic cleavage of the t-butyl ester of 21 was effected by treatment with BCl₃, and the crude trihydroxy acid was subject to lactonization-chlorination to complete the total synthesis of salinosporamide A.

In certain embodiments, the synthesis of salinosporamide A or analogues thereof staring from a readily available pyrroglutamate derivatve include the following steps:

(a) providing a pyrroglutamate derivative of formula: 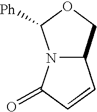
wherein Ph is phenyl or a substituted phenyl;

(b) reacting the pyrroglutamate derivative derivative with a vinyl nucleophile or other aliphatic nucleophile to generate a compound of formula:

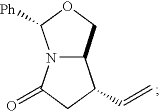

(c) alkylating the resulting compound at C-2 to yield a compound of formula: 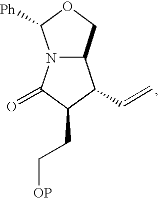
wherein P is an oxygen-protecting group;

(d) oxonolysing followed by reductive treatment of the carbon-carbon double bond to yield a primary alcohol of formula:

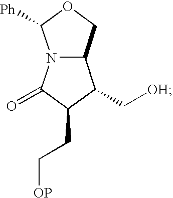

(e) reacting the primary alcohol with ClCO₂R, wherein R is C₁-C₆ alkyl, preferably ethyl, to yield an ethyl carbonate of formula:

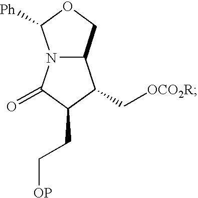

(f) removing the N,O-acetal protecting group by treatment with acid;

(g) oxidizing the resulting primary alcohol to yield the carboxylic acid of formula:

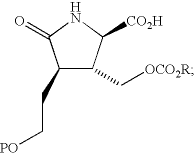

(h) esterifying the resulting carboxylic acid;

(i) treating the resulting ester with Meerwein reagent to yield a compound of formula: 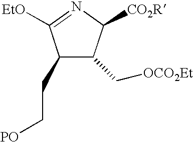
wherein R′ is C₁-C₆ alkyl, preferably t-butyl;

(j) treating the compound with a base to form the lactone of formula:

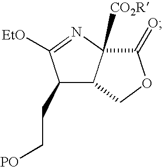

(k) treating the lactone with acid to afford the lactam;

(l) protecting the nitrogen of the lactam;

(m) removing the oxygen-protecting group to yield the compound of formula: 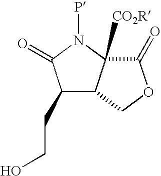
wherein P′ is a nitrogen protecting group, preferably PMB;

(n) opening the lactone ring with phenylselenium anion to yield a compound of formula:

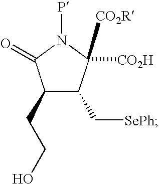

(o) protecting the carboxylic acid functional group;

(p) oxidizing the selenide and unprotected alcohol to yield a compound of formula:

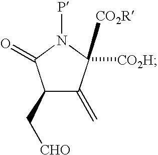

(q) cyclizing the compound to form an intermediate hemiacetal of formula:

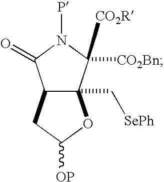

(r) deselenylating via a radical reaction to yield a compound of formula:

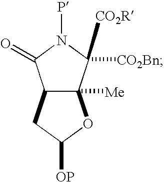

(s) reducing and oxidizing the benzyl ester to form the aldehyde of formula:

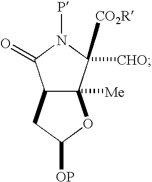

(t) treating the aldehyde with a cyclohexenyl zinc reagent to yield a compound of formula:

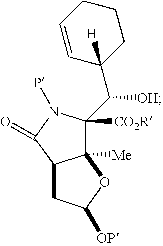

(u) removing the nitrogen-protecting group;

(v) opening the glycoside to yield the triol of formula:

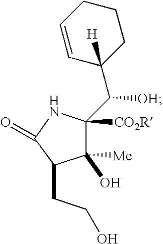

(w) hydrolyzing the alkylester;

(x) lactonizing under suitable conditions to form the 4-membered lactone; and

(y) chlorinating the primary alcohol to yield salinosporamide A:

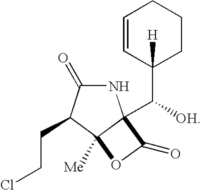

In certain embodiments, a pyrroglutamate derivative of the formula: 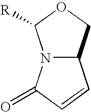
wherein R is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl, preferably phenyl or substituted phenyl;

is reacted with a nucleophile (e.g., metal reagent such as a vinyl cuprate reagent, zinc reagent, magnesium reagent, lithium reagent) under suitable conditions to yield a 1,4-addition product of the formula: 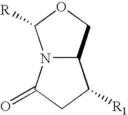
wherein R₁ is as defined above; and R₁ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; preferably, vinyl; and

the 1,4-addition product is reacted with a base (e.g., LDA) to form the enolate which is then reacted with an electrophile (e.g., alkyl iodide, alkyl tosylate, alkyl mesylate) to yield an alkylation product of the formula: 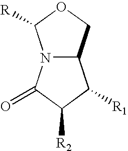
wherein R and R₁ are as defined above, and R₂ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl, preferably —CH₂CH₂OP, wherein P is hydrogen or an oxygen-protecting group. In certain embodiments, the 1,4-addition proceeds from the α-face. In certain embodiments, the alkylation proceeds from the β-face. In certain particular embodiments, the 1,4-addition proceeds from the α-face, and the alkylation proceeds from the β-face yielding the stereochemistry of the product as shown above. In certain other embodiments, the 1,4-addition proceeds from the β-face, and the alkylation proceeds from the α-face yielding the opposite stereochemistry than shown in the formula above.

The product of the 1,4-addition and the alkylation provides a useful intermediate in synthesizing salinosporamide A and its analogues. The invention provides an intermediate of formula: 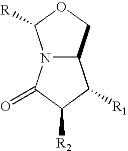
wherein R, R₁, and R₂ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl. In certain embodiments, R is phenyl or a substituted phenyl. In certain embodiments, R is phenyl. In certain embodiments, R₁ is vinyl. In certain embodiments, R₁ is ethyl, —CH₂OAc, —CH₂OH, —CH₂OP, —CH₂OCO₂R, —CH₂OCO₂Et, —CHO, —CH₂I, —CH₂Cl, —CH₂Br, or —CH₂NR₂, wherein P is an oxygen-protecting group, and R is hydrogen, aliphatic, heteroaliphatic, aryl, or heteroaryl. In other embodiments, R₂ is —CH₂CH₂OP, wherein P is an oxygen protecting group (e.g., silyl protecting groups, benzyl, alkyl, etc.), preferably benzyl.

In certain embodiments, a lactone of formula: 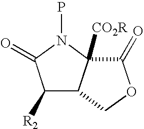
wherein R is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; preferably, R is 1-butyl;

R₂ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

P is a nitrogen protecting group, preferably PMB; is reacted with phenylselenium anion or thiolate under suitable conditions to yield the carboxylic acid: 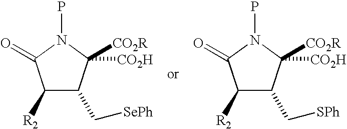
wherein R, R₂, and P are defined as above. In certain embodiments, other selenium or sulfur reagents besides phenylselenium anion or phenylthiolate are used, for example, a substituted phenylselenium anion or a substituted phenylthiolate may be used or an alkylselenium anion or alkylthiolate may be used. The selenium anion may be produced using a reducing agent such as sodium borohydride. In certain embodiments, R is t-butyl. In certain embodiments, P is PMB. In certain embodiments, R₂ is —CH₂CH₂OH. The carboxylic acid or the proctected form of formula: 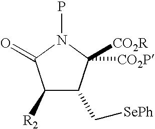
is a useful intermediate in the synthesis of salinosporamide A and its analogues. The carboxylic acid or the proctected form of formula: 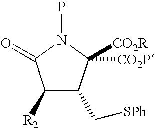
is also a useful intermediate in the synthesis of salinosporamide A and its analogues.

In certain embodiments, the intermediate of formula: 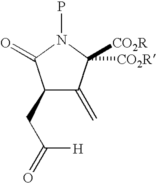
wherein R and R′ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

P is a nitrogen-protecting group, preferably PMB;
is subjected to an acetal-mediated cationic cycliczation. In certain embodiments, the acetal-mediated cationic cyclization is accomplished under conditions including phenylselenenyl bromide and AgBF₄ to yield the cyclization product: 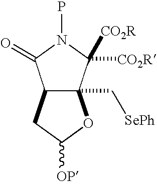
wherein R and R′ are defined above; P is a nitrogen-protecting group, preferably PMB; and P′ is an oxygen-protecting group, preferably benzyl. The cyclization may then be deselenenylated to provide the methyl functionality at C3. The deselenenylation may be accomplished using AIBN and n-Bu₃SnH. The deselenenylation reaction yields an intermediate of formula: 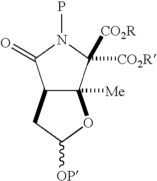
wherein R, R′, P, and P′ are as defined above. In certain other embodiments, the selenium-containing moiety of the exo-cyclic double is coverted to another functional to yield a compound of the formula: 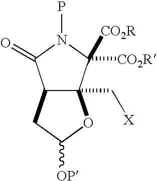
where in X is aliphatic, heteroaliphatic, hydroxyl, alkoxy, alkylthioxy, arylthioxy, halogen, amino, alkylamino, dialkylamino, cyano, acyl, aryl, or heteroaryl. In certain embodiments, X is hydroxyl, alkoxy, halogen, alkylthioxy, amino, alkylamino, or dialkylamino.

In certain embodiments, the intermediate is of the formula: 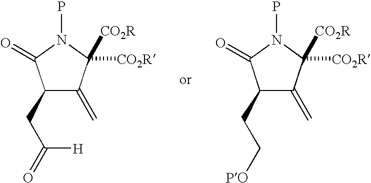
wherein R and R′ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl;

P is a nitrogen-protecting group, preferably PMB; and

P′ is hydrogen or an oxygen-protecting group. In certain embodiments, R′ is benzyl. In other embodiments, R is t-butyl.

In some embodiments, analogues of salinosporamide A are synthesized by modification of certain steps in the synthesis described above. First, the alkylation of 6 by forming the enolate followed by reaction with an electrophile may be performed using various electrophiles. For example, the electrophile may be a aliphatic or heteroaliphatic iodide. For substituted electrophile, functional groups may be protected to prevent undesired reaction. In one embodiment, a compound of formula: 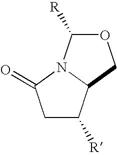
wherein R and R′ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl;

is reacted with a reagent of formula:
R₃—X
wherein R₃ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

X is halogen, —OTs, or other leaving group;
under suitable conditions to form a compound of formula: 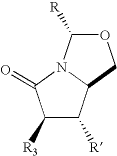
wherein R, R′, and R₃ are defined as above. The reaction may be accomplished by treating the starting material with a base such as LDA, and reacting the resulting enolate with an electrophile such as an alkyl halide.

Second, N-alkylation of the amide may be accomplished with any electrophile such as an alkyl halide. In certain embodiments, this allows one to replace the amide hydrogen with an aliphatic, heteroaliphatic, or acyl group. In one embodiment, a compound of formula: 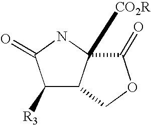
wherein R is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R₃ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

is reacted with a reagent of formula:
R₁—X
wherein R₁ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —OR_{A}; ═O; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

X is halogen, —OTs, or other leaving group.
under suitable conditions to form a compound of formula: 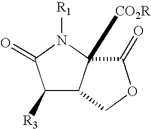
wherein R, R₁, and R₃ are defined as above.

Third, rather than reacting the bicyclic aldehyde 18 with the cyclohexenyl zinc reagent another nucleophile may be used. In certain embodiments, the nucleophile is a zinc reagent. In other embodiments, the nucleophile may be a lithium, copper, Grignard, or other metal reagent. Preferably, the reagent allows for stereoselective installation of the aliphatic, heteroaliphatic, aryl, or heteroaryl group. The aliphatic, heteroaliphatic, aryl, or heteroaryl group may be substituted or unsubstituted. In certain embodiments, a compound of formula: 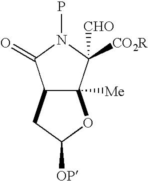
wherein R, P, and P′ are independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl;

is reacted with a nucleophile of formula:
^{⊖R}_{B}′
wherein R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —OR_{B}″; ═O; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; under suitable conditions to form a compound of formula: 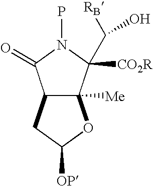
wherein R, P, P′, and R_{B}′ are defined as above. In certain embodiments, the nucleophile is a zinc reagent.
Pharmaceutical Compositions

This invention also provides a pharmaceutical preparation comprising at least one of the compounds as described above and herein, or a pharmaceutically acceptable derivative thereof, which compounds inhibit the growth of or kill tumor cells. In other embodiments, the compounds show cytostatic or cytotoxic activity against neoplastic cells such as cancer cells. In yet other embodiments, the compounds inhibit the growth of or kill rapidly dividing cells such as stimulated inflammatory cells. In certain other embodiments, the compounds are anti-microbial compound.

As discussed above, the present invention provides novel compounds having antimicrobial and/or antiproliferative activity, and thus the inventive compounds are useful for the treatment of a variety of medical conditions including infectious diseases, cancer, autoimmune diseases, inflammatory diseases, and diabetic retinopathy. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents, e.g., another anti-microbial agent or another anti-proliferative agent. In other embodiments, these compositions further comprise an anti-inflammatory agent such as aspirin, ibuprofen, acetaminophen, etc., pain reliever, or anti-pyretic. In other embodiments, these compositions further comprise an anti-emetic agent, a pain reliever, a multi-vitamin, etc.

It will also be appreciated that certain of the compounds of the present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977; incorporated herein by reference. The salts can be prepared in situ during the final isolation and purification of the compounds of the invention, or separately by reacting the free base functionality with a suitable organic or inorganic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates. In certain embodiments, the esters are cleaved by enzymes such as esterases.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutical Compositions

The invention further provides a method of treating infections and inhibiting tumor growth. The method involves the administration of a therapeutically effective amount of the compound or a pharmaceutically acceptable derivative thereof to a subject (including, but not limited to a human or animal) in need of it.

The compounds and pharmaceutical compositions of the present invention may be used in treating or preventing any disease or conditions including proliferative diseases (e.g., cancer, benign neoplasms, diabetic retinopathy), and autoimmune diseases (e.g., rheumatoid arthritis, lupus). The compounds and pharmaceutical compositions may be administered to animals, preferably mammals (e.g., domesticated animals, cats, dogs, mice, rats), and more preferably humans. Any method of administration may be used to deliver the compound of pharmaceutical compositions to the animal. In certain embodiments, the compound or pharmaceutical composition is administered orally. In other embodiments, the compound or pharmaceutical composition is administered parenterally.

The exact amount required will vary from subject to subject, depending on the species, age, and general condition of the subject, the particular compound, its mode of administration, its mode of activity, and the like. The compounds of the invention are preferably formulated in dosage unit form for ease of administration and uniformity of dosage. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracistemally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such an Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Total Synthesis of Salinosporamide A

In Scheme 1 below, there is described the overall stereochemical strategy for the synthesis of salinosporamide A. The strong facial bias of the pyrroglutamate derivative, 3, served to direct attack at C₃ (originally conducted by 1,4-addition of a vinyl cuprate nucleophile) from its α-face. Correspondingly, alkylation at C₂ proceeds with high selectivity from its β-face. The α-substituent, introduced at C₃, in time is presented as a carbonate ester. To enable the strategic C-acylation, a novel imidate ensemble (see formal structure 4) was devised to direct lithiation to C₄. Following intramolecular acylation by the carbonate ester, as practiced in our recent synthesis of jiadifenin (Cho et al. J. Am. Chem. Soc. 126:14358, 2004; incorporated herein by reference), a structurally differentiated malonate moiety is created with complete stereochemical definition. In time, the substituent at C₃ is presented as an exo-methylene group (cf. 4→5). An acetaldehyde residue, derivable at C₂, is used to differentiate the faces of this exo-methylene group (cf. 5→6), thereby ensuring the properly configured β-lactone moiety. Adaptation of the Corey concept in the context of addition of the allylic zinc reagent 8 (Reddy et al. J. Am. Chem. Soc. 126:6230, 2004; incorporated herein by reference) to constrained aldehyde 7 provides remarkable stereoselection at both C₆ and C₅.

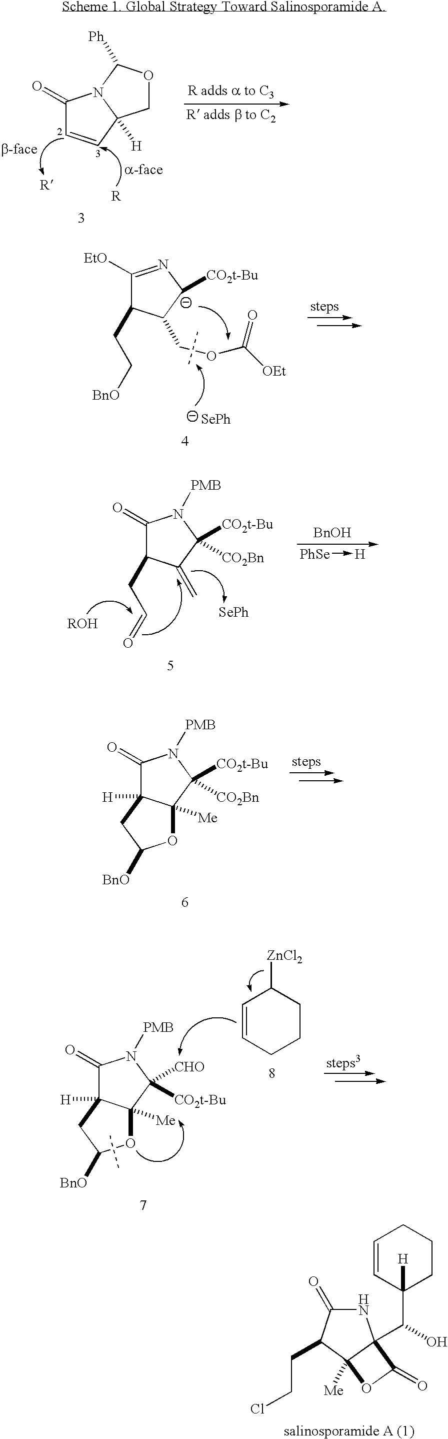

The bicyclic enamide 3 (Thottathil et al. J. Org. Chem. 51:3140, 1986; Hamada et al. J. Am. Chem. Soc. 111:1524, 1989; Hamada et al. Tetrahedron 47:8635, 1991; each of which is incorporated herein by reference) was treated with divinyl cuprate under mediation by TMSCl (Corey et al. Tetrahedron Lett. 26:6019, 1985; incorporated herein by reference), affording 9 as a single product (Scheme 2). In a subsequent step, alkylation of 9, as shown, furnished the lactam 11 in 77% yield as a 14:1 mixture of diastereomers (For earlier studies of stereocontrolled access to all-trans a,b,g-substituted pyrrolidinone from 3, see: (a) Hanessian et al. Synlett 1990, 501. (b) Baldwin et al. Tetrahedron Lett. 1991, 32, 1379. (c) Okamoto et al. Tetrahedron: Asymmetry 2001, 12, 1353; each of which is incorporated herein by reference). We next turned to the conversion of the vinyl group to a carbonate ester acylating agent. Ozonolysis followed by reductive treatment with sodium borohydride afforded 12. The derived ethyl carbonate was subjected to cleavage of the N,O-acetal protecting arrangement to afford 13. The hydroxymethyl lactam was converted to the imidate ester 4 as shown, by a sequence consisting of Jones' oxidation, esterification, and treatment with Meerwein reagent (Et₃OBF₄). With the lactam functionality thus masked, treatment of 4 with LHMDS led to exclusive anion formation at C₄. Internal acylation with the pendant ethylcarbonate proceeded smoothly to afford lactone 14 (Cho et al. J. Am. Chem. Soc. 126:14358, 2004; incorporated herein by reference). Acidic treatment of 14 led to the restoration of the lactam moiety, which was subsequently protected with PMBCl. Removal of the benzyl protecting group afforded 15.

The lactone of 15 was subjected to nucleophilic ring opening with phenylselenium anion (Scarborough et al. J. Am. Chem. Soc. 102:3904, 1980; incorporated herein by reference) and the resultant carboxylic acid was benzylated to afford the differentially esterified 16 (Scheme 3). Surprisingly, the subsequent selenide oxidation elimination sequence gave rise to a mixture of the expected alcohol 17 (72%), along with aldehyde 5 (22%), which was in fact a one-step advancement in our planned synthetic route. Upon purification, we converted the bulk unoxidized material, 17, to aldehyde 5 through exposure to Dess-Martin periodinane (Dess, D. B.; Martin, J. C. J. Am. Chem. Soc. 1982, 104, 902; incorporated herein by reference).

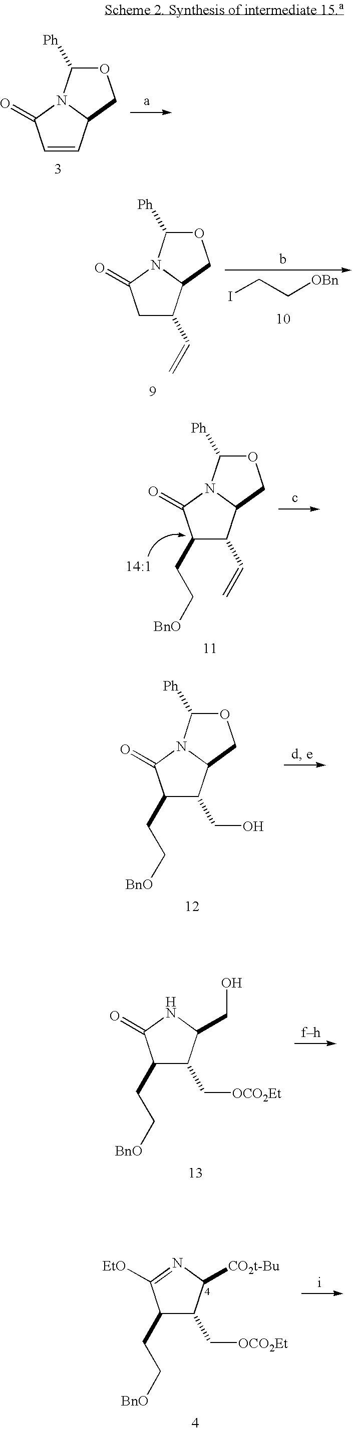

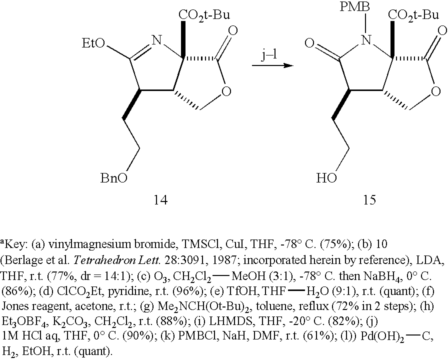

With intermediate 5 in hand, the stage was now set for a key acetal-mediated cationic cyclization (Current et al. Tetrahedron Lett. 51:5075, 1978; incorporated herein by reference). We note that electrophilically induced cyclization at the aldehyde (or hemiacetal) oxidation level was central to the success of the project. Thus, a tetrahydrofuran derived from haloetherification could not have been readily opened to expose the required functionalities at C₂ and C₃. Conversely, selenolactonization using an acetic acid residue at C₂ would have produced a lactone which would not be readily differentiable from the bis-acyl functionality already present at C₄. Thus, recourse to the benzyl glycoside modality for storing and unveiling the C₂-C₃ functionality was a unique solution to a difficult problem. Upon treatment with phenylselenenyl bromide and AgBF₄ in the presence of benzyl alcohol, an intermediate hemiacetal was generated, which presumably assisted in the phenylselenenylation of the exocyclic methylene to afford 18. Importantly, this reaction allowed for the introduction of the quaternary center at C₃ with complete stereoselectivity. Radical deselenenylation provided the desired methyl functionality at C₃. Upon conversion of the benzyl ester to an aldehyde, intermediate 7 was in hand.

Treatment of 7 with the cyclohexenyl zinc reagent, 8, under the Corey protocol (Reddy et al. J. Am. Chem. Soc. 126:6230, 2004; incorporated herein by reference), proceeded with excellent diastereocontrol to afford 19 in 88% yield (dr=20:1 at C₆) (The use of the corresponding imidate aldehyde instead of 7 resulted in poor diastereoselectivity (78% yield, 4:3, configuration not determined). Obviously, the PMB group places a critical role in diastereoselection). Removal of the PMB group, followed by reductive opening of the benzyl glycoside gave rise to triol 20. Acidic cleavage of the t-butyl ester was effected through treatment with BCl₃ and the crude trihydroxy acid was then subjected to lactonization-chlorination (Reddy et al. J. Am. Chem. Soc. 126:6230, 2004; incorporated herein by reference) to provide salinosporamide A (1), whose spectroscopic properties were in complete accord with the natural material (Feling et al. Angew. Chem. Int. Ed. 42:355, 2003; incorporated herein by reference).

In summary, an efficient and highly stereocontrolled enantioselective synthesis of salinosporamide A has been achieved. Several key features of our synthesis include the temporary masking of a lactam functionality to accomplish selective anion formation at C₄ (see 4), the use of a nucleophilic selenium species to open a lactone in a regiocontrolled fashion (see 15), and the use of an unusual cationic hemiacetal selenocyclization to install the quaternary center at C₃ in manageable form with complete stereocontrol. 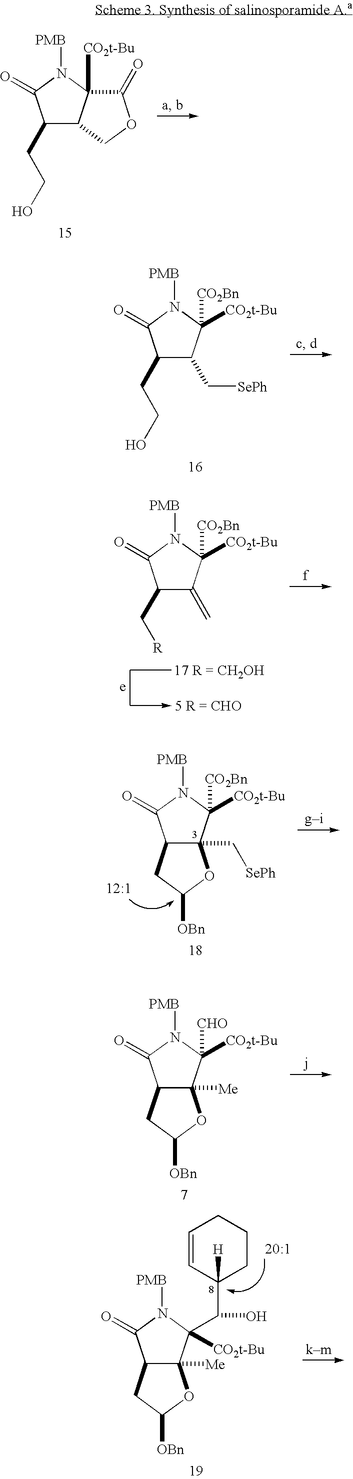 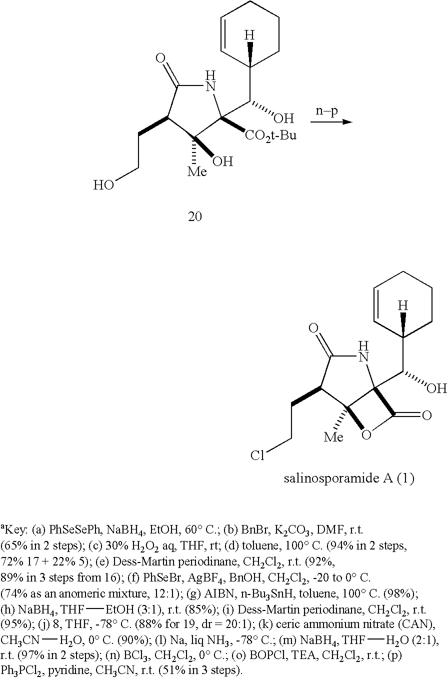
Experimentals 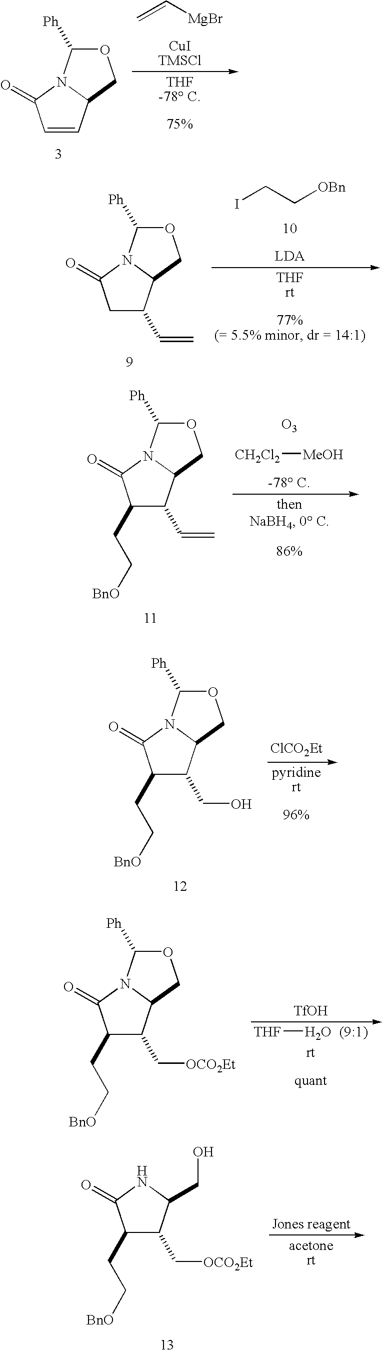 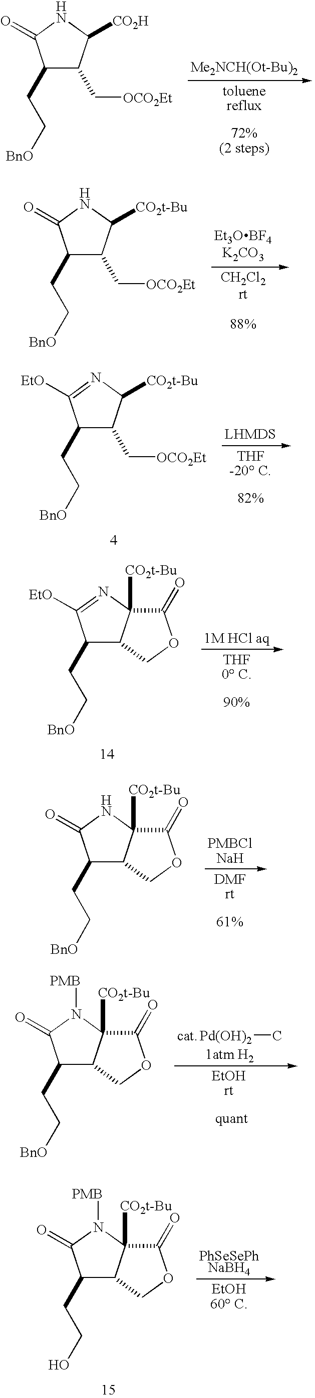 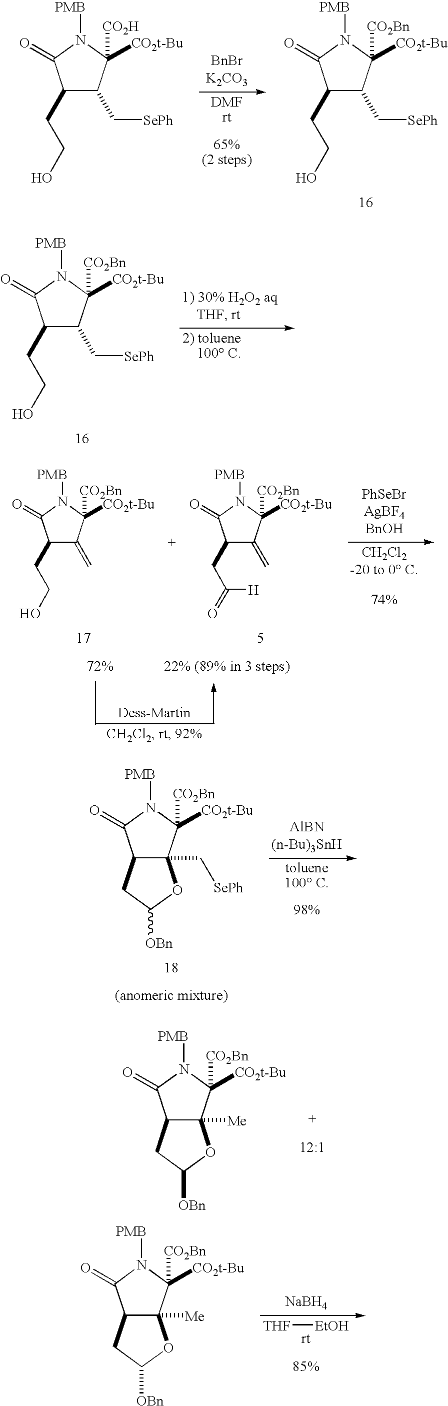 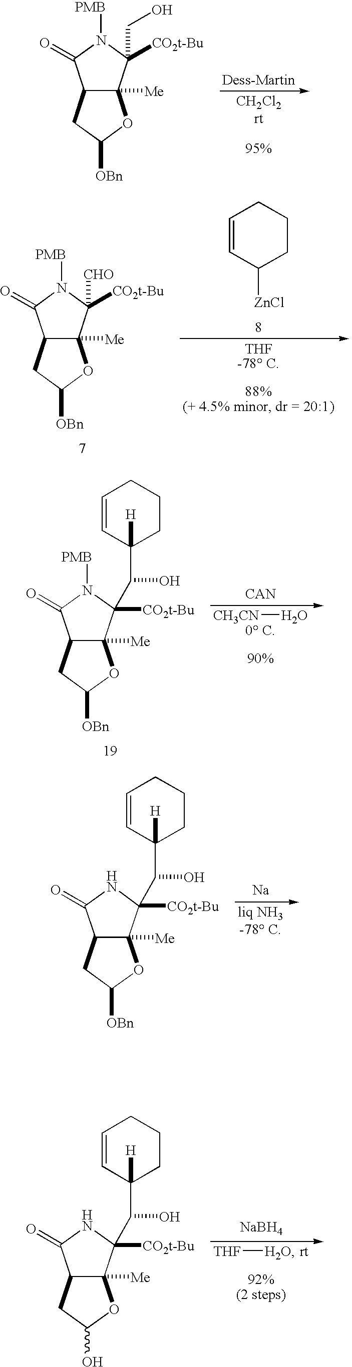 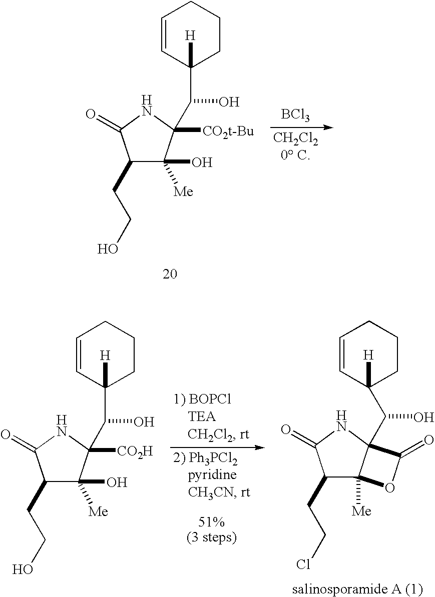
Experimental Details

General Considerations: All glassware was dried in an oven at 150° C. prior to use. All air/moisture sensitive experiments were conducted under a slight static pressure of dry Ar unless indicated otherwise. Anhydrous benzene, toluene, Et₂O, CH₂Cl₂, THF were obtained using Solv-Tek, Inc. solvent purification system. All other solvents were of anhydrous quality purchased from Aldrich Chemicals Co. All chemicals were purchased from Aldrich Chemical Co. and used as received. Commercial grade solvents were used for routine purposes without further purification. Pyridine, triethylamine (TEA), (i-Pr)₂NH, and TMSCl were distilled from CaH₂ under a N₂ atmosphere prior to use. All NMR spectra were recorded on a Bruker model AMX-400 (¹H: 400 MHz, ¹³C: 100 MHz) or a Bruker model DRX-500 (¹H: 500 MHz, ¹³C: 125 MHz) NMR spectrometer. Chemical shifts are reported in parts per million (ppm) from internal tetramethylsilane or the residual solvent signal of CDCl₃. Spectra were taken in CDCl₃ unless noted otherwise. The following abbreviations were used in reporting spectra: s=singlet, d=doublet, t=triplet, m=multiplet, dd=double doublet, ddd=double double doublet, br=broad, brs=broad singlet. Infrared spectra were taken on a Perkin Elmer 1600 Series FTIR Spectrometer using thin neat film deposition on NaCl plates and peaks are reported in wave numbers (cm⁻¹). High resolution mass spectra (HRMS) were taken on a Micromass Q-TOF Ultima. Column chromatography was performed with Merck silica gel 60 (40-63 mesh).

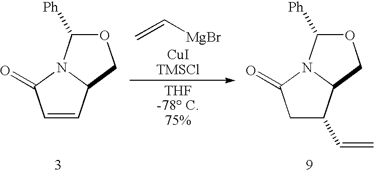

Vinyl lactam 9. Vinylmagnesium bromide (1M solution in THF, 320 ml, 320 mmol) was added to a slurry of CuI (30.5 g, 160 mmol) in THF (300 ml) at −20° C. and the resultant mixture was stirred at this temperature for 1 h. The vinyl cuprate thus prepared was cooled to −78° C. and a mixture of the enamide 3 (21.45 g, 107 mmol) and TMSCl (27.0 ml, 213 mmol) in THF (150 ml) was added slowly. After stirring at −78° C. for 2 hours, the reaction was quenched with saturated NH₄Cl solution, diluted with EtOAc, and filtered through a pad of Celite. The filtrate was sequentially washed with 1M HCl solution, saturated NaHCO₃ solution, and saturated NaCl solution. The organic layer was concentrated in vacuo and the residue was purified by silica gel column chromatography (40% EtOAc in hexanes) to give the vinyl lactam 9 (18.37 g, 80.1 mmol, 75%) as a yellow oil.

¹H NMR (400 MHz, CDCl₃)

δ 7.42-7.34 (m, 2H), 7.34-7.22 (m, 3H), 6.32 (s, 1H), 5.80 (ddd, 1H, J=17.4, 10.2, 7.6 Hz), 5.15-5.02 (m, 2H), 4.12 (dd, 1H, J=8.4, 6.5 Hz), 3.88 (dd, 1H, J=13.2, 6.5 Hz), 3.63 (dd, 1H, J=8.4, 6.5 Hz), 2.87 (m, 1H), 2.74-2.55 (m, 2H).

¹³C NMR (100 MHz, CDCl₃)

δ 176.99, 138.61, 137.59, 129.00, 128.85, 126.40, 117.34, 87.62, 70.90, 64.29, 45.58, 40.95.

FTIR (neat) νₘₐₓ: 3032, 2916, 1709, 1375, 1351, 1248, 1219, 1175, 1026, 923, 741, 699.

HRMS (ESI) m/z calcd for C₁₄H₁₆NO₂ [M+H]⁺: 230.1181, found 230.1188.

[α]²³_{D}-169 (c 1.6, CHCl₃).

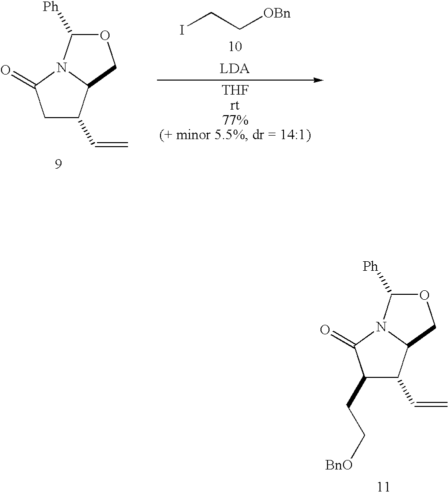

Benzyloxyethyl lactam 11. n-BuLi (2.5 M solution in hexanes, 20.3 ml, 50.8 mmol) was added to a solution of (i-Pr)₂NH in THF (100 ml) at 0° C. and the resultant mixture was stirred for 45 min. A solution of the lactam 9 (10.10 g, 44.1 mmol) in THF (50 ml) was added to the freshly prepared LDA solution at 0° C. After stirring for 1 h, the benzyloxyethyl iodide 10 (Berlage et al. Tetrahedron Lett. 1987, 28, 3091; incorporated herein by reference) (19.2 ml, 116 mmol) was added and the mixture was warmed up to room temperature. After stirring for 2 h, the reaction was quenched with saturated NH₄Cl solution and partitioned between EtOAc and saturated NaCl solution. The organic layer was concentrated in vacuo and the residue was purified by silica gel column chromatography (40% EtOAc in hexanes) to give the benzyloxyethyl lactam 11 (12.27 g, 33.8 mmol, 77%) as a yellow oil and its diastereomer (885 mg, 2.44 mmol, 5.5%).

¹H NMR (400 MHz, CDCl₃)

δ 7.39-7.33 (m, 2H), 7.33-7.19 (m, 8H), 6.30 (s, 1H), 5.75 (ddd, 1H, J=17.1, 10.3, 8.4 Hz), 5.09 (d, 1H, J=17.1 Hz), 5.06 (d, 1H, J=10.3 Hz), 4.43 (s, 2H), 4.06 (dd, 1H, J=8.7, 6.5 Hz), 3.78 (m, 1H), 3.66-3.59 (m, 3H), 2.84 (m, 1H), 2.50 (m, 1H), 2.03 (m, 1H), 1.71 (m, 1H).

¹³C NMR (100 MHz, CDCl₃)

δ 178.38, 138.92, 138.66, 137.34, 129.00, 128.96 (2C), 128.96 (2C), 128.10 (2C), 127.96, 126.50 (2C), 118.37, 87.61, 73.23, 70.84, 67.84, 62.23, 53.98, 47.35, 29.18.

FTIR (neat) νₘₐₓ: 2859, 1706, 1455, 1356, 1214, 1098, 1026, 924, 735, 698.

HRMS (ESI) m/z calcd for C₂₃H₂₆NO₃ [M+H]⁺; 364.1913, found 364.1926.

[α]²³_{D}-85.0 (c 1.2, CHCl₃).

Data for the diastereomer of 11 (minor product)

¹H NMR (400 MHz, CDCl₃)

δ 7.43-7.38 (m, 2H), 7.37-7.25 (m, 8H), 6.37 (s, 1H), 5.83 (ddd, 1H, J=17.1, 10.3, 8.3 Hz), 5.16 (d, 1H, J=10.3 Hz), 5.11 (d, 1H, J=17.1 Hz), 4.52 (d, 1H, J=11.7 Hz), 4.47 (d, 1H, J=11.7 Hz), 4.14 (dd, 1H, J=8.3, 6.6 Hz), 3.98 (m, 1H), 3.70-3.61 (m, 3H), 2.95 (m, 1H), 2.85 (m, 1H), 1.97 (m, 2H).

¹³C NMR (125 MHz, CDCl₃)

δ 179.42, 138.09, 138.04, 134.18, 128.18, 128.10 (2C), 128.02 (2C), 127.43 (2C), 127.23, 125.67 (2C), 117.98, 86.94, 72.70, 69.46, 67.39, 61.47, 47.23, 46.58, 27.46.

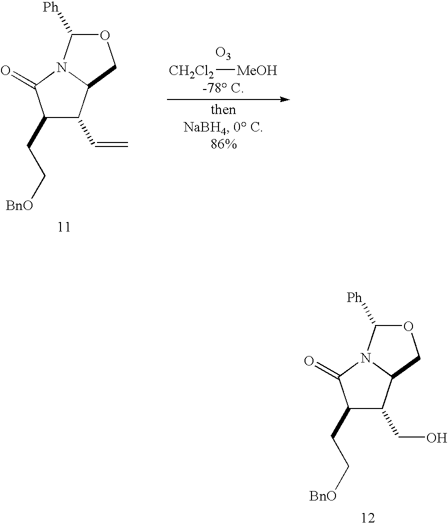

Alcohol 12. A solution of the benzyloxyethyl lactam 11 (62.2 mg, 0.171 mmol) in CH₂Cl₂-MeOH (3:1, 5.0 ml) was treated with 03 at −78° C. After complete consumption of the substrate, excess O₃ was removed by N₂ bubbling and the mixture was treated with NaBH₄ (52.2 mg, 1.38 mmol). After stirring at −78° C. for 30 min, the mixture was warmed up to 0° C. and stirred for additional 30 min. The reaction mixture was diluted with EtOAc and sequentially washed with 1M citric acid solution and saturated NaCl solution. The organic layer was concentrated in vacuo and the residue was purified by silica gel column chromatography (80% EtOAc in hexanes) to give the alcohol 12 (53.8 mg, 0.146 mmol, 86%) as a colorless oil.

¹H NMR (400 MHz, CDCl₃)

δ 7.40-7.32 (m, 2H), 7.32-7.20 (m, 8H), 6.78 (s, 1H), 4.47 (d, 1H, J=12.2 Hz), 4.42 (d, 1H, J=12.2 Hz), 4.15 (dd, 1H, J=8.3, 6.5 Hz), 3.79 (dd, 1H, J=13.4, 6.7 Hz), 3.72 (m, 1H), 3.68-3.55 (m, 3H), 3.52 (m, dd, J=8.3, 7.0 Hz), 2.76 (m, 1H), 2.32 (t, 1H, J=5.5 Hz), 2.22 (m, 1H), 2.16 (m, 1H).

¹³C NMR (100 MHz, CDCl₃)

δ 178.65, 138.83, 138.13, 128.97, 128.92 (2C), 128.82 (2C), 128.32, 128.29 (2C), 126.43 (2C), 87.40, 73.61, 71.94, 69.01, 64.27, 60.27, 50.07, 45.54, 30.00.

FTIR (neat) νₘₐₓ: 3438, 2872, 1700, 1455, 1359, 1271, 1176, 1098, 1027, 742, 699.

HRMS (ESI) m/z calcd for C₂₂H₂₆NO₄ [M+H]⁺: 368.1862, found 368.1863.

[α]²³_{D}-85.0 (c 0.60, CHCl₃).

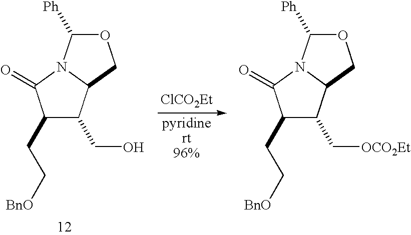

Carbonate. The alcohol 12 (12.68 g, 34.5 mmol) was dissolved in pyridine (50 ml) and treated with ClCO₂Et (4.00 ml, 41.8 mmol) at 0° C. The reaction mixture was allowed to warm up to room temperature and stirred for 12 h. The mixture was treated with additional ClCO₂Et (3.30 ml, 34.5 mmol) and stirring was continued for additional 7 h. The reaction mixture was diluted with Et₂O and poured into 2M H₂SO₄ solution. The organic layer was sequentially washed with saturated NaHCO₃ solution and saturated NaCl solution, and then concentrated in vacuo. The residue was purified by silica gel column chromatography (40% EtOAc in hexanes) to give the carbonate (14.61 g, 33.2 mmol, 96%) as a pale yellow oil.

¹H NMR (400 MHz, CDCl₃)

δ 7.38-7.35 (m, 2H), 7.32-7.19 (m, 8H), 6.25 (s, 1H), 4.44 (s, 2H), 4.32 (dd, 1H, J=11.0, 4.3 Hz), 4.17 (dd, 1H, J=8.4, 5.5 Hz), 4.15-4.07 (m, 3H), 3.84 (dd, 1H, J=13.4, 6.7 Hz), 3.61 (t, 2H, J=5.5 Hz), 3.51 (dd, 1H, J=8.3, 7.1 Hz), 2.77 (m, 1H), 2.35 (m, 1H), 2.13 (m, 1H), 1.74 (m, 1H), 1.23 (t, 3H, J=7.1 Hz).

¹³C NMR (100 MHz, CDCl₃)

δ 177.70, 155.24, 138.69, 138.57, 129.02, 128.84 (2C), 128.80 (2C), 128.11 (2C), 128.03, 126.42 (2C), 87.42, 73.42, 71.94, 68.57, 68.15, 64.76, 60.85, 46.21, 44.91, 30.06, 14.64.

FTIR (neat) νₘₐₓ: 2871, 1745, 1701, 1456, 1356, 1262, 1094, 1027, 1007, 746, 700.

HRMS (ESI) m/z calcd for C₂₅H₃₀NO₆ [M+H]⁺: 440.2073, found 440.2089.

[α]²³_{D}-63.9 (c 1.8, CHCl₃).

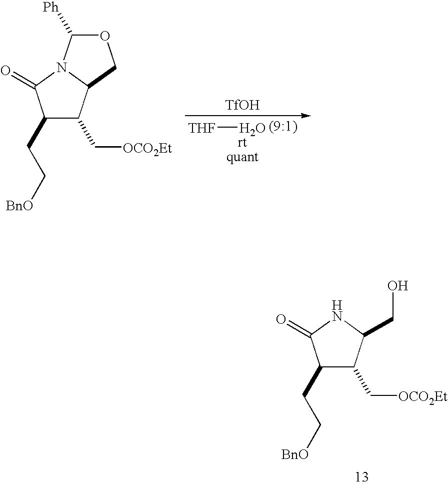

Hydroxymethyl lactam 13. The carbonate (14.61 g, 33.2 mmol) was dissolved in THF—H₂O (9:1, 400 ml) and treated with TfOH (13.5 ml, 155 mmol) at room temperature. After stirring for 18 h, the reaction mixture was diluted with EtOAc and poured into cooled saturated NaHCO₃ solution. The organic layer was washed with saturated NaCl solution and concentrated in vacuo. The residue was purified by silica gel column chromatography (10% MeOH in CHCl₃) to give the hydroxymethyl lactam 13 (11.68 g, 33.2 mmol, quantitative yield) as a yellow oil.

¹H NMR (400 MHz, CDCl₃)

δ 7.28-7.17 (m, 5H), 6.59 (s, 1H), 4.43 (s, 2H), 4.19 (dd, 1H, J=11.0, 4.6 Hz), 4.10 (m, 2H), 3.67 (dd, 1H, J=11.1, 2.9 Hz), 3.56 (m, 2H), 3.49 (m, 1H), 3.36 (dd, 1H, J=11.1, 6.6 Hz), 2.89 (br, 1H), 2.36 (m, 1H), 2.23 (m, 1H), 2.08 (m, 1H), 1.72 (m, 2H), 1.23 (t, 3H, J=7.1 Hz).

¹³C NMR (100 MHz, CDCl₃)

δ 178.84, 155.35, 138.60, 128.77 (2C), 128.08 (2C), 127.98, 73.34, 68.55, 68.28, 65.57, 64.68, 57.93, 41.80, 41.74, 30.96, 14.64.

FTIR (neat) νₘₐₓ:3280, 2871, 1745, 1684, 1456, 1368, 1262, 1092, 1007, 746, 700.

HRMS (ESI) m/z calcd for C₁₈H₂₆NO₆ [M+H]⁺: 352.1760, found 352.1774.

[α]²³_{D}-9.2 (c 1.2, CHCl₃).

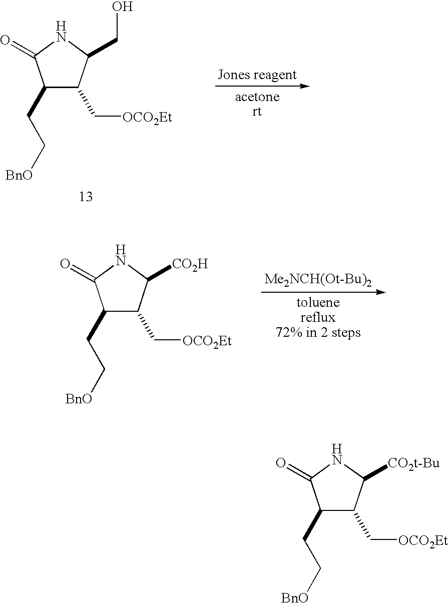

t-Butyl Ester. Jones reagent (70 ml) was added to a solution of the hydroxymethyl lactam 13 (11.68 g, 33.2 mmol) in acetone (210 ml) at 0° C. and the resultant mixture was allowed to warm up to room temperature. After 3.5 h, 2-propanol (15 ml) was added to quench excess reagent and stirring was continued for additional 30 min. The mixture was partitioned between EtOAc and saturated NaCl solution. The organic layer was further washed with saturated NaCl solution and concentrated in vacuo. The crude carboxylic acid thus obtained was dissolved in toluene, mixed with N,N-dimethylformamide di-tert-butyl acetal (25 ml, 0.10 mol) at room temperature, and heated to reflux for 1 h. After cooling down, the mixture was partitioned between EtOAc and 5% NaCl solution. The organic layer was further washed with saturated NaCl solution and concentrated in vacuo. The residue was purified by silica gel column chromatography (50% EtOAc in hexanes) to give the t-butyl ester (10.08 g, 23.9 mmol, 72% in 2 steps) as an orange oil.

¹H NMR (400 MHz, CDCl₃)

d 7.29-7.09 (m, 5H), 6.01 (br, 1H), 4.43 (s, 2H), 4.27 (d, 1H, J=4.7 Hz), 4.13 (d, 1H, J=7.1 Hz), 4.09 (d, 1H, J=7.1 Hz), 3.90 (d, 1H, J=6.8 Hz), 3.57 (t, 2H, J=6.1 Hz), 2.58 (m, 1H), 2.44 (m, 1H), 2.08 (m, 1H), 1.74 (m, 1H), 1.64 (br, 1H), 1.39 (9H), 1.23 (t, 3H, J=7.1 Hz).

¹³C NMR (100 MHz, CDCl₃)

177.94, 170.74, 155.32, 138.71, 128.71 (2C), 127.93 (2C), 127.88, 83.15, 73.28, 68.35, 67.41, 64.63, 56.57, 44.25, 41.22, 30.76, 28.30 (3C), 14.60.

FTIR (neat) νₘₐₓ:2980, 1743, 1700, 1456, 1368, 1258, 1158, 1009.

HRMS (ESI) m/z calcd for C₂₂H₃₂NO₇ [M+H]⁺: 422.2179, found 422.2191.

[α]²³_{D}-8.3 (c 0.63, CHCl₃).

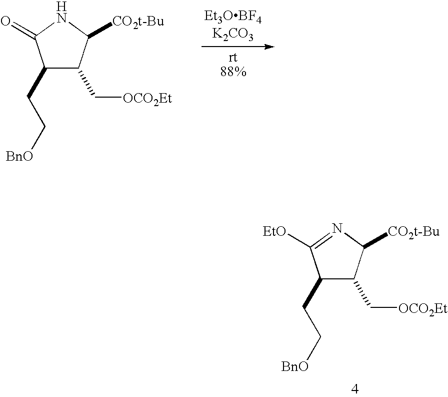

Imidate 4. A mixture of the t-butyl ester (9.90 g, 23.5 mmol) and powdered K₂CO₃ (13.0 g, 94.2 mmol) in CH₂Cl₂ (250 ml) was treated with Et₃OBF₄ (8.93 g, 47.0 mmol) at 0° C. and the reaction was allowed to warm up to room temperature. After stirring for 4 h, the reaction mixture was poured into saturated NaHCO₃ solution and extracted with EtOAc. The organic layer was washed with saturated NaCl solution and concentrated in vacuo. The residue was purified by silica gel column chromatography (50% EtOAc in hexanes) to give the imidate 4 (9.33 g, 20.8 mmol, 88%) as a yellow oil.

¹H NMR (400 MHz, CDCl₃)

δ 7.31-7.17 (m, 5H), 4.43 (s, 2H), 4.25-4.05 (m, 7H), 3.57-3.46 (m, 2H), 2.69 (m, 1H), 2.48 (m, 1H), 2.02 (m, 1H), 1.69 (m, 1H), 1.40 (s, 9H), 1.30-1.19 (m, 6H).

¹³C NMR (100 MHz, CDCl₃)

δ 174.73, 172.79, 155.30, 138.67, 128.60 (2C), 127.89 (2C), 127.81, 81.37, 73.18, 69.84, 68.83, 68.10, 64.71, 64.29, 44.51, 31.91, 28.32, 28.26 (3C), 14.55, 14.53.

FTIR (neat) νₘₐₓ:2979, 1745, 1652, 1456, 1368, 1258, 1154, 1098, 1029, 876, 791, 699.

HRMS (ESI) m/z calcd for C₂₄H₃₆NO₇ [M+H]⁺: 450.2492, found 450.2506.

[α]²³_{D}-13.8 (c 2.9, CHCl₃).

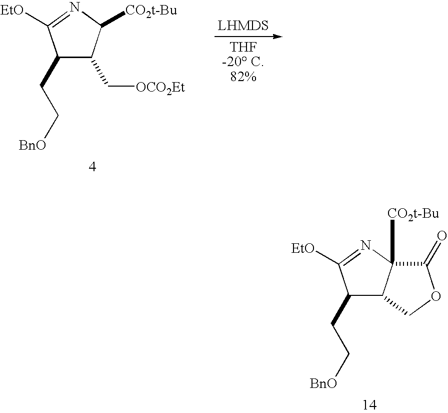

Lactone 14. A solution of the imidate 4 (2.71 g, 6.03 mmol) in THF (70 ml) was treated with LHMDS (1M in THF, 7.84 ml, 7.84 mmol) at −20° C. After stirring for 10 min, the reaction was quenched with saturated NH₄Cl solution and partitioned between EtOAc and saturated NaCl solution. The organic layer was concentrated in vacuo and the residue was purified by silica gel column chromatography (40% EtOAc in hexanes) to give the lactone 14 (2.00 g, 4.96 mmol, 82%) as a yellow oil.

¹H NMR (400 MHz, CDCl₃)

δ 7.36-7.20 (m, 5H), 4.42 (s, 2H), 4.35-4.24 (m, 2H), 4.35 (dd, 1H, J=9.5, 7.4 Hz), 4.02 (dd, 1H, J=9.5, 2.5 Hz), 3.55-3.45 (m, 2H), 3.02 (m, 1H), 2.78 (m, 1H), 2.08 (m, 1H), 1.61 (m, 1H), 1.40 (s, 9H), 1.24 (t, 3H, J=7.1 Hz).

¹³C NMR (100 MHz, CDCl₃)

δ 176.64, 172.66, 168.31, 138.25, 128.86 (2C), 128.25, 128.10 (2C), 83.67, 79.64, 73.57, 71.71, 68.55, 66.18, 50.62, 50.35, 31.63, 28.22 (3C), 14.55.

FTIR (neat) νₘₐₓ: 2978, 2934, 2869, 1783, 1747, 1630, 1478, 1456, 1370, 1333, 1258, 1158, 1026, 835, 746, 700.

HRMS (ESI) m/z calcd for C₂₂H₃₀NO₆ [M+H]⁺: 404.2073, found 404.2090.

[α]²³_{D} 28.9 (c 1.9, CHCl₃).

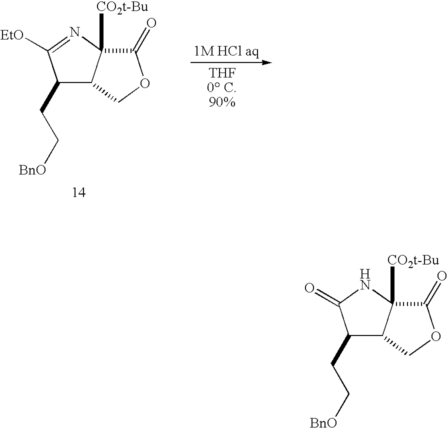

Lactam. A solution of the lactone 14 (2.10 g, 5.20 mmol) in THF (30 ml) was treated with 1M aqueous HCl (10 ml) at 0° C. After stirring for 1 h, the reaction mixture was neutralized with saturated NaHCO₃ solution and extracted with EtOAc. The organic layer was washed with saturated NaCl solution and concentrated in vacuo. The residue was purified by silica gel column chromatography (50% EtOAc in hexanes) to give the lactam (1.77 g, 4.72 mmol, 90%) as a yellow solid.

¹H NMR (400 MHz, CDCl₃)

δ 7.40-7.23 (m, 5H), 6.38 (s, 1H), 4.47 (s, 2H), 4.44 (dd, 1H, J=9.6, 6.6 Hz), 4.23 (dd, 1H, J=9.6, 1.7 Hz), 3.66 (m, 1H), 3.58 (m, 1H), 3.14 (m, 1H), 2.52 (m, 1H), 2.27 (m, 1H), 1.72 (m, 1H), 1.47 (s, 9H).

¹³C NMR (100 MHz, CDCl₃)

δ 176.39, 172.55, 166.45, 138.13, 128.92 (2C), 128.32, 128.14 (2C), 85.61, 73.66, 72.31, 69.12, 67.05, 47.97, 46.53, 30.97, 28.17 (3C).

FTIR (neat) νₘₐₓ: 2979, 1783, 1749, 1634, 1456, 1371, 1334, 1253, 1158, 1026, 845, 746, 700, 668.

HRMS (ESI) m/z calcd for C₂₀H₂₆NO₆ [M+H]⁺: 376.1760, found 376.1777.

[α]²³_{D} 16.6 (c 0.61, CHCl₃).

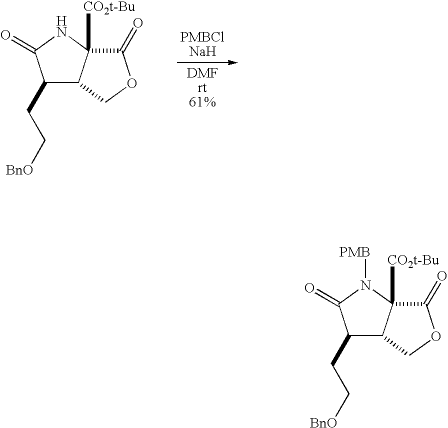

N-PMB lactam. A mixture of the lactam (395 mg, 1.05 mmol) and p-methoxybenzyl chloride (PMBCl, 450 μl, 3.32 mmol) in DMF (4.5 ml) was treated with NaH (60% in mineral oil, 84 mg, 5.8 mmol) at 0° C. and the reaction was allowed to warm up to room temperature. After stirring for 3 h, the reaction was quenched with H₂O and partitioned between Et₂O and H₂O. The organic layer was washed with saturated NaCl solution and concentrated in vacuo. The residue was purified by silica gel column chromatography (20% EtOAc in hexanes) to give the N-PMB lactam (322 mg, 0.650 mmol, 62%) as a pale yellow oil.

¹H NMR (400 MHz, CDCl₃)

δ 7.30-7.17 (m, 7H), 6.73 (d, 2H, J=8.7 Hz), 4.69 (d, 1H, J=5.2 Hz), 4.45-4.35 (m, 4H), 3.93 (dd, 1H, J=9.5, 5.3 Hz), 3.70 (s, 3H), 3.62-3.50 (m, 2H), 3.11 (m, 1H), 2.44 (m, 1H), 2.12 (m, 1H), 1.64 (m, 1H), 1.33 (s, 9H).

¹³C NMR (100 MHz, CDCl₃)

δ 175.99, 170.90, 167.27, 159.24, 138.33, 130.41 (2C), 129.17, 128.89 (2C), 128.26, 128.10 (2C), 114.02 (2C), 85.20, 73.59, 71.83, 71.05, 68.93, 55.58, 46.46, 46.11, 45.53, 31.46, 28.12 (3C).

FTIR (neat) νₘₐₓ: 2934, 2359, 1783, 1734, 1706, 1615, 1513, 1456, 1371, 1248, 1152, 1094, 1028, 835, 748, 700, 668.

HRMS (ESI) m/z calcd for C₂₈H₃₄NO₇ [M+H]⁺: 496.2335, found 496.2342.

[α]²³_{D} 21.6 (c 0.88, CHCl₃).

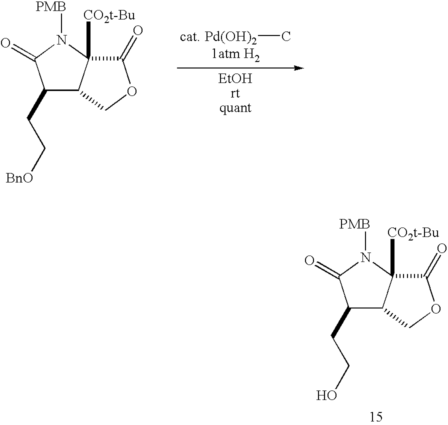

Alcohol 15. A mixture of the N-PMB lactam (670 mg, 1.35 mmol) and Pd(OH)₂ (20% on carbon, 60.8 mg) in EtOH (30 ml) was vigorously stirred for 16 hours under 1 atm hydrogen atmosphere. The catalyst was filtered off through a Celite pad and the filtrate was concentrated in vacuo to give the alcohol 15 (550 mg, 1.35 mmol, quantitative yield) as a colorless oil.

¹H NMR (400 MHz, CDCl₃)

δ 7.22 (d, 2H, J=8.9 Hz), 6.73 (d, 2H, J=8.9 Hz), 4.72 (d, 1H, J=15.2 Hz), 4.58 (dd, 1H, J=9.5, 8.2 Hz), 4.42 (d, 1H, J=15.2 Hz), 4.01 (dd, 1H, J=9.5, 5.9 Hz), 3.80-3.72 (m, 2H), 3.71 (s, 3H), 3.19 (m, 1H), 2.53 (m, 1H), 2.38 (br, 1H), 1.92 (m, 1H), 1.70 (m, 1H), 1.40 (s, 9H)

¹³C NMR (100 MHz, CDCl₃).

δ 176.73, 170.69, 167.15, 159.27, 130.25 (2C), 128.90, 114.06 (2C), 85.46, 71.69, 71.26, 60.88, 55.58, 46.47, 46.12, 45.01, 33.95, 28.11 (3C).

FTIR (neat) νₘₐₓ: 3458, 2935, 1784, 1734, 1690, 1684, 1652, 1615, 1514, 1456, 1395, 1372, 1280, 1250, 1152, 1034, 835, 753, 668.

HRMS (ESI) m/z calcd for C₂₁H₂₈NO₇ [M+H]⁺: 406.1866, found 406.1885.

[α]²³_{D} 67.3 (c 0.52, CHCl₃).

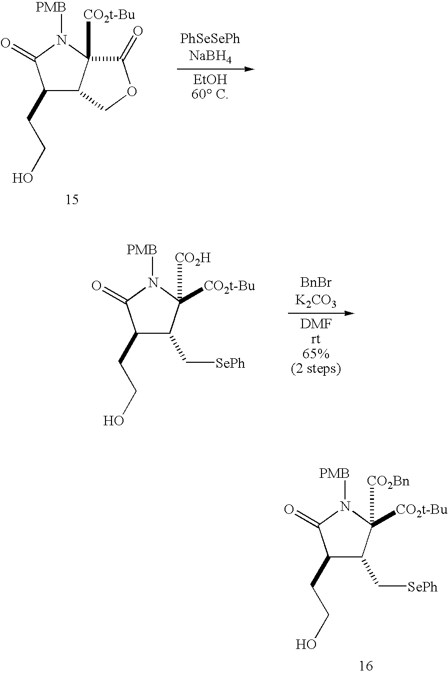

Benzyl ester 16. NaBH₄ (370 mg, 9.78 mmol) was added to a previously degassed (vacuum-pump-thaw) mixture of the alcohol 15 (990 mg, 2.44 mmol) and PhSeSePh (1.53 g, 4.90 mmol) in EtOH (20 ml) at room temperature. After 1 h, the mixture was heated to 60° C. and stirring was continued for 3 h. The mixture was concentrated and partitioned between 1M KOH solution and Et₂O. The aqueous layer was acidified with 1M citric acid solution and extracted with EtOAc. The organic layer was washed with saturated NaCl solution and concentrated in vacuo. The crude product (yellow solid, 1.20 g) was used for the next reaction without purification.

A mixture of the crude carboxylic acid (1.20 g) and powdered K₂CO₃ (3.37 g, 24.4 mmol) in DMF (15 ml) was treated with BnBr (1.16 ml, 9.75 mmol) at room temperature. After stirring for 15 h, the reaction mixture was diluted with EtOAc and sequentially washed with 5% NaCl solution and saturated NaCl solution. The organic layer was concentrated in vacuo and the residue was purified by silica gel column chromatography (50% EtOAc in hexanes) to give the benzyl ester 16 (1.03 g, 1.58 mmol, 65% in 2 steps) as a pale yellow oil.

¹H NMR (400 MHz, CDCl₃)

δ 7.37-7.32 (m, 2H), 7.32-7.15 (m, 8H), 6.98 (d, 2H, J=8.7 Hz), 6.72 (d, 2H, J=8.7 Hz), 5.07 (d, 1H, J=8.0 Hz), 4.90 (d, 1H, J=8.0 Hz), 4.75 (d, 1H, J=15.8 Hz), 4.35 (d, 1H, J=15.8 Hz), 3.97 (dd, 1H, J=8.5, 2.8 Hz), 3.68 (s, 3H), 3.60 (m, 1H), 3.46 (m, 1H), 3.10 (dd, 1H, J=12.5, 5.1 Hz), 3.02 (m, 1H), 2.87 (dd, 1H, J=12.5, 7.0 Hz), 2.52 (dt, 1H, J=9.8, 3.2 Hz), 1.87 (m, 1H), 1.70 (m, 1H), 1.06 (s, 9H).

¹³C NMR (100 MHz, CDCl₃)

δ 178.90, 168.03, 165.58, 159.14, 134.73, 133.05 (2C), 130.40, 129.74 (2C), 129.38 (2C), 129.34, 129.20, 129.13 (2C), 128.50 (2C), 127.82, 114.21 (2C), 84.91, 75.63, 68.72, 61.75, 55.69, 46.30, 46.14, 45.83, 33.72, 27.82, 27.59 (3C).

FTIR (neat) νₘₐₓ: 3393, 2933, 1732, 1700, 1684, 1652, 1616, 1513, 1456, 1437, 1394, 1370, 1290, 1248, 1150, 1034, 836, 739, 693, 668.

HRMS (ESI) m/z calcd for C₃₄H₄₀NO₇Se [M+H]⁺: 654.1970, found 654.1959.

[α]²³_{D}-1.1 (c 0.88, CHCl₃).

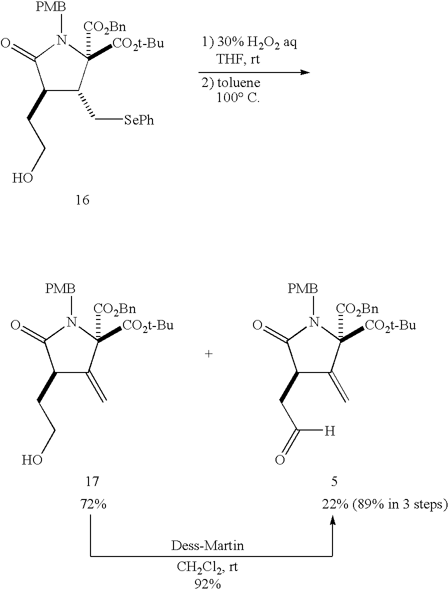

Ene-ol 17. A solution of the benzyl ester 16 (1.03 g, 1.58 mmol) in THF (25 ml) was treated with 30% aqueous H₂O₂ solution (2.50 ml) at room temperature and stirring was continued until complete consumption of the substrate (˜4 h). The reaction mixture was cooled to 0° C., carefully quenched with saturated Na₂SO₃ solution, and extracted with EtOAc. The organic layer was washed with saturated NaCl solution and concentrated in vacuo to give the corresponding selenoxide as a yellow oil, which was dissolved in toluene (60 ml) and stirred at 100° C. for 2 h. After cooling down, the reaction mixture was diluted with EtOAc, sequentially washed with saturated NaHCO₃ solution and saturated NaCl solution, and concentrated in vacuo. The residue was purified by silica gel column chromatography (50-70% EtOAc in hexanes) to give the ene-ol 17 (565 mg as a pale yellow oil, 1.14 mmol, 72%) and the ene-al 5 (174 mg as a pale yellow oil, 0.35 mmol, 22%).

Ene-al 5. A solution of the ene-ol 17 (565 mg, 1.14 mmol) in CH₂Cl₂ (15 ml) was treated with Dess-Martin periodinane (580 mg, 1.37 mmol) at room temperature. After stirring for 1.5 h, 2-propanol (0.5 ml) was added to the reaction mixture to quench excess reagent, and stirring was continued for additional 30 min. The reaction mixture was diluted with Et₂O, filtered through a pad of Celite, and concentrated in vacuo. The residue was purified silica gel column chromatography (80% Et₂O in hexanes) to give the ene-al 5 (520 mg, 1.05 mmol, 92%, 694 mg in total, 89% in 3 steps from the benzyl ester 16) as a pale yellow oil.

Data for Ene-ol 17

¹H NMR (400 MHz, CDCl₃)

δ 7.30-7.23 (m, 3H), 7.16-7.10 (m, 2H), 7.02 (d, 2H, J=8.6 Hz), 6.72 (d, 2H, J=8.6 Hz), 5.54 (d, 1H, J=2 Hz), 5.33 (d, 1H, J=2 Hz), 4.83 (d, 1H, J=12.2 Hz), 4.73 (d, 1H, J=15.9 Hz), 4.67 (d, 1H, J=12.2 Hz), 4.47 (d, 1H, J=15.9 Hz), 3.83-3.72 (m, 2H), 3.69 (s, 3H), 3.34-3.26 (m, 2H), 2.01 (m, 1H), 1.91 (m, 1H), 2.20 (s, 9H).

¹³C NMR (100 MHz, CDCl₃)

δ 177.49, 167.11, 166.01, 159.13, 141.09, 134.96, 129.09, 128.96 (2C), 128.89 (2C), 128.71 (2C), 115.01, 114.18 (2C), 84.64, 76.37, 68.27, 61.02, 55.67, 46.06, 44.32, 35.34, 27.77 (3C).

FTIR (neat) νₘₐₓ: 3393, 2933, 1731, 1700, 1684, 1652, 1615, 1513, 1456, 1437, 1394, 1369, 1290, 1248, 1150, 1034, 836, 739, 693, 668.

HRMS (ESI) m/z calcd for C₂₈H₃₄NO₇ [M+H]⁺: 496.2335, found 496.2341.

[α]²³_{D} 16.9 (c 1.3, CHCl₃).

Data for ene-al 5

¹H NMR (400 MHz, CDCl₃)

δ 9.72 (s, 1H), 7.28-7.23 (m, 3H), 7.15-7.08 (m, 2H), 7.04 (d, 2H, J=8.6 Hz), 6.72 (d, 2H, J=8.6 Hz), 5.51 (d, 1H, J=2.5 Hz), 5.27 (d, 1H, J=2.5 Hz), 4.80 (d, 1H, J=12.2 Hz), 4.78 (d, 1H, J=15.9 Hz), 4.68 (d, 1H, J=12.2 Hz), 4.47 (d, 1H, J=15.9 Hz), 3.70 (s, 3H), 3.64 (m, 1H), 3.00 (dd, 1H, J=18.2, 4.4 Hz), 2.80 (dd, 1H, J=18.2, 7.0 Hz), 1.21 (s, 9H).

¹³C NMR (100 MHz, CDCl₃)

δ 199.27, 175.35, 167.20, 165.63, 159.12, 140.69, 134.97, 129.20, 128.99, 128.96 (4C), 128.67 (2C), 115.26, 114.14 (2C), 84.57, 75.98, 68.26, 55.66, 46.16, 45.60, 39.79, 27.79 (3C).

FTIR (neat) νₘₐₓ: 2977, 1727, 1710, 1662, 1613, 1514, 1456, 1394, 1370, 1280, 1249, 1220, 1177, 1152, 1031, 995, 921, 836, 750, 699.

HRMS (ESI) m/z calcd for C₂₈H₃₂NO₇ [M+H]⁺: 494.2179, found 494.2178.

[α]²³_{D} 8.1 (c 0.98, CHCl₃).

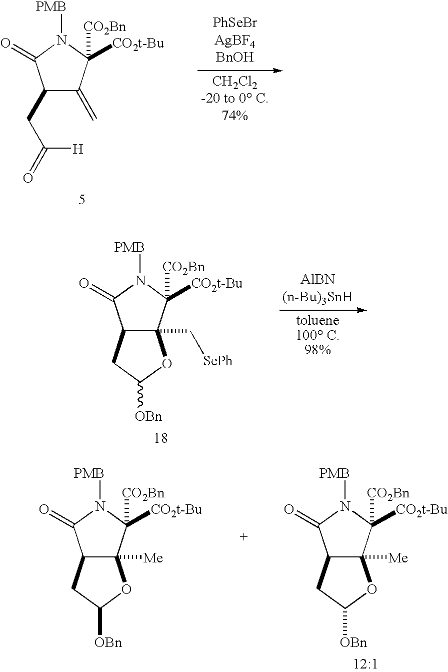

Benzyl glycoside 18. A mixture of the ene-al 5 (355 mg, 0.712 mmol), BnOH (298 ml, 2.88 mmol), and AgBF₄ (560 mg, 2.88 mmol) in CH₂Cl₂ (35 ml) was treated with PhSeBr (680 mg, 2.88 mmol) at −20° C. After 30 min, the reaction was allowed to warm up to 0° C. and stirred for additional 1.5 h. The reaction mixture was poured into a 2:2:1 mixture of saturated NaCl, saturated NaHCO₃, and saturated Na₂SO₃ (25 ml) and extracted with EtOAc. The whole mixture was filtered through a pad of Celite and the organic layer was separated. Concentration in vacuo followed by purification of the residue by silica gel column chromatography (20% EtOAc in hexanes) gave the benzyl glycoside 18 (pale yellow oil, 403 mg, 0.533 mmol, 74%) as an inseparable anomeric mixture (12:1, determined after the next reaction).

Data for the Major Isomer

¹H NMR (500 MHz, CDCl₃)

δ 7.31-7.04 (m, 17H), 6.61 (d, 2H, J=8.6 Hz), 5.09 (s, 1H, J=5.3 Hz), 4.94 (d, 1H, J=14.9 Hz), 4.57 (d, 1H, J=11.9 Hz), 4.49 (d, 1H, J=14.9 Hz), 4.44 (d, 1H, J=12.1 Hz), 4.36 (d, 1H, J=12.1 Hz), 4.16 (d, 1H, J=11.9 Hz), 3.71 (d, 1H, J=13.3 Hz), 3.63 (s, 3H), 3.09 (d, 1H, J=13.3 Hz), 2.87 (d, 1H, J=8.8 Hz), 2.70 (m, 1H), 2.39 (d, 1H, J=13.0 Hz), 1.42 (s, 9H).

¹³C NMR (125 MHz, CDCl₃)

δ 176.30, 168.28, 164.71, 159.20, 138.14, 134.51, 132.22 (2C), 131.12 (2C), 130.71, 129.56 (2C), 129.17, 129.09 (2C), 128.88 (2C), 128.73, 128.65 (2C), 128.30 (2C), 127.80, 127.42, 113.78 (2C), 104.17, 91.44, 83.77, 79.04, 69.41, 68.27, 55.60, 50.16, 45.75, 38.23, 33.16, 28.28 (3C).

FTIR (neat) νₘₐₓ: 2957, 2359, 1747, 1707, 1613, 1576, 1512, 1456, 1438, 1392, 1368, 1297, 1246, 1178, 1155, 1103, 1017, 950, 838, 7346, 696.

HRMS (ESI) m/z calcd for C₄₁H₄₄NO₈Se [M+H]⁺: 758.2232, found 758.2227.

Methyl lactam. A mixture of benzyl glycoside 18 (403 mg, 0.533 mmol, as an anomeric mixture), AIBN (8.7 mg, 0.053 mmol), and n-Bu₃SnH (350 μl, 1.32 mmol) in toluene (6.0 ml) was heated to 100° C. After stirring for 2 h, the reaction mixture was cooled down and directly subjected to purification by silica gel column chromatography (30-40% EtOAc in hexanes) to give the methyl lactam (white solid, 290 mg, 0.482 mmol, 90%) and its anomeric isomer (white solid, 24.0 mg, 0.039 mmol, 7.5%).

Data for the Major Isomer

¹H NMR (400 MHz, CDCl₃)

δ 7.35-7.11 (m, 8H), 7.25 (d, 2H, J=8.6 Hz), 7.11-7.03 (m, 2H), 6.63 (d, 2H, J=8.6 Hz), 5.05 (d, 1H, J=5.0 Hz), 5.01 (d, 1H, J=14.9 Hz), 4.59 (d, 1H, J=12.0 Hz), 4.54 (d, 1H, J=14.9 Hz), 4.42 (d, 1H, J=12.1 Hz), 4.32 (d, 1H, J=12.1 Hz), 4.18 (d, 1H, J=12.0 Hz), 3.64 (s, 3H), 2.83 (d, 1H, J=8.0 Hz), 2.51 (d, 1H, J=13.3 Hz), 2.10 (ddd, 1H, J=13.3, 8.0, 5.0 Hz), 1.44 (s, 9H), 1.33 (s, 3H).

¹³C NMR (100 MHz, CDCl₃)

δ 75.77, 167.82, 164.69, 158.69, 137.73, 134.23, 130.75 (2C), 128.50, 128.47 (2C), 128.43, 128.29 (2C), 128.13 (2C), 127.72 (2C), 127.24, 113.26 (2C), 101.71, 88.52, 82.82, 77.83, 68.35, 67.46, 55.09, 49.91, 45.03, 34.92, 27.77 (3C), 21.25.

FTIR (neat) νₘₐₓ: 2944, 1746, 1706, 1653, 1616, 1558, 1512, 1456, 1393, 1300, 1248, 1159, 1097, 1024, 993, 920, 838, 750, 697.

HRMS (ESI) m/z calcd for C₃₅H₄₀NO₈ [M+H]⁺: 602.2754, found 602.2751.

[α]²³_{D}-25.3 (c 0.53, CHCl₃).

Data for the Minor Isomer

¹H NMR (500 MHz, CDCl₃)

δ 7.29-7.19 (m, 8H), 7.16 (d, 2H, J=8.7 Hz), 7.04-7.09 (m, 2H), 6.71 (d, 2H, J=8.7 Hz), 5.18 (dd, 1H, J=5.9, 4.4 Hz), 5.09 (d, 1H, J=14.9 Hz), 4.64 (d, 1H, J=11.5 Hz), 4.52 (d, 1H, J=14.9 Hz), 4.36 (d, 1H, J=12.1 Hz), 4.35 (d, 1H, J=11.5 Hz), 4.31 (d, 1H, J=12.1 Hz), 2.97 (d, 1H, J=4.4 Hz), 2.69 (ddd, 1H, J=14.0, 6.1, 1.1 Hz), 2.19 (ddd, 1H, J=13.8, 9.2, 4.3 Hz), 1.51 (s, 3H), 1.44 (s, 9H).

¹³C NMR (125 MHz, CDCl₃)

δ 176.09, 167.18, 164.44, 158.94, 137.49, 134.25, 130.92 (2C), 128.58, 128.55 (2C), 128.43 (2C), 128.29 (2C), 128.22, 127.84 (2C), 127.76, 113.45 (2C), 103.69, 87.73, 83.15, 77.21, 70.11, 67.56, 55.21, 51.01, 45.02, 35.00, 27.93 (3C), 20.47.

HRMS (ESI) m/z calcd for C₃₅H₄₀NO₈ [M+H]⁺: 602.2754, found 602.2750.

[α]²³_{D} 21.5 (c 2.5, CHCl₃).

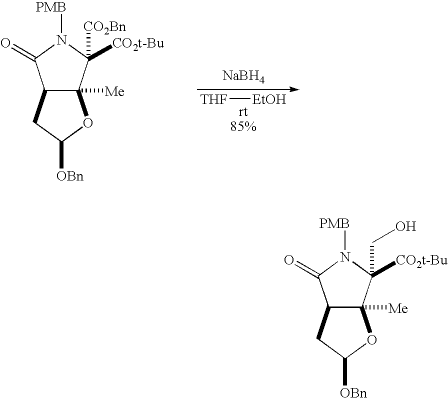

Alcohol. A solution of the methyl lactam (major isomer, 290 mg, 0.482 mmol) in THF-EtOH (3:1, 6.0 ml) was treated with NaBH₄ (110 mg, 2.91 mmol) at room temperature. After stirring for 6 h, the reaction mixture was diluted with EtOAc and sequentially washed with 1M citric acid solution and saturated NaCl solution. The organic layer was concentrated in vacuo and the residue was purified by silica gel column chromatography (80% EtOAc in hexanes) to give the alcohol (204 mg, 0.410 mmol, 85%) as a white foam.

¹H NMR (400 MHz, CDCl₃)

δ 7.32 (d, 2H, J=8.6 Hz), 7.30-7.11 (m, 5H), 6.72 (d, 2H, J=8.6 Hz), 5.15 (d, 1H, J=15.2 Hz), 5.03 (d, 1H, J=5.1 Hz), 4.65 (d, 1H, J=12.0 Hz), 4.33 (d, 1H, J=15.2 Hz), 4.20 (d, 1H, J=12.0 Hz), 3.74 (dd, 1H, J=12.8, 10.2 Hz), 3.71 (s, 3H), 3.26 (dd, 1H, J=12.8, 5.2 Hz), 2.67 (d, 1H, J=8.2 Hz), 2.49 (d, 1H, J=13.2 Hz), 2.12 (ddd, 1H, J=13.2, 8.2, 5.2 Hz), 1.48 (s, 9H), 1.40 (s, 3H), 1.01 (br, 1H).

¹³C NMR (100 MHz, CDCl₃)

δ 176.10, 166.84, 159.01, 138.18, 130.80, 129.51 (2C), 128.17 (2C), 127.76 (2C), 127.20, 114.26 (2C), 101.67, 88.61, 82.35, 78.10, 68.61, 61.57, 55.19, 50.58, 44.60, 34.81, 28.11 (3C), 21.02.

FTIR (neat) νₘₐₓ: 33330, 2980, 1741, 1717, 1700, 1684, 1653, 1616, 1558, 1540, 1514, 1456, 1368, 1246, 1159, 1101, 1026, 920, 800, 735, 668.

HRMS (ESI) m/z calcd for C₂₈H₃₆NO₇ [M+H]⁺: 498.2492, found 498.2495.

[α]²³_{D}-77.0 (c 0.40, CHCl₃).

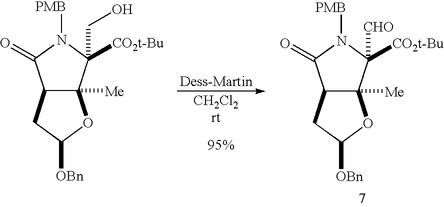

Aldehyde 7. A solution of the alcohol (168 mg, 0.338 mmol) in CH₂Cl₂ (8.0 ml) was treated with Dess-Martin periodinane (186 mg, 0.439 mmol) at room temperature. After stirring for 1.5 h, 2-propanol (0.1 ml) was added to quench excess reagent and stirring was continued for additional 30 min. The mixture was diluted with Et₂O, filtered through a pad of Celite, and concentrated in vacuo. The residue was purified by silica gel column chromatography (80% Et₂O in hexanes) to give the aldehyde 7 (159 mg, 0.321 mmol, 95%) as a white foam.

¹H NMR (400 MHz, CDCl₃)

δ 9.34 (s, 1H), 7.27-7.22 (m, 5H), 7.08 (d, 2H, J=8.7 Hz), 6.58 (d, 2H, J=5.8 Hz), 5.09 (d, 1H, J=5.2 Hz), 4.70 (d, 1H, J=14.6 Hz), 4.68 (d, 1H, J=12.3 Hz), 4.42 (d, 1H, J=14.6 Hz), 4.27 (d, 1H, J=12.3 Hz), 3.64 (s, 3H), 2.71 (d, 1H, J=8.3 Hz), 2.53 (d, 1H, J=13.4 Hz), 2.14 (ddd, 1H, J=13.4, 8.3, 5.2 Hz), 1.52 (s, 9H), 1.30 (s, 3H).

¹³C NMR (100 MHz, CDCl₃)

δ 196.67, 174.89, 164.51, 158.90, 137.74, 130.64 (2C), 128.53, 128.23 (2C), 127.65 (2C), 127.32, 113.62 (2C), 102.37, 88.64, 83.93, 81.95, 68.74, 55.10, 49.70, 45.67, 34.79, 28.09 (3C), 21.78.

FTIR (neat) νₘₐₓ: 2940, 1700, 1653, 1616, 1514, 1456, 1395, 1301, 1248, 1155, 1099, 1024, 917, 810, 750, 699, 668.

HRMS (ESI) m/z calcd for C₂₈H₃₄NO₇ [M+H]⁺: 496.2335, found 496.2361.

[α]²³_{D}-36.5 (c 0.34, CHCl₃).

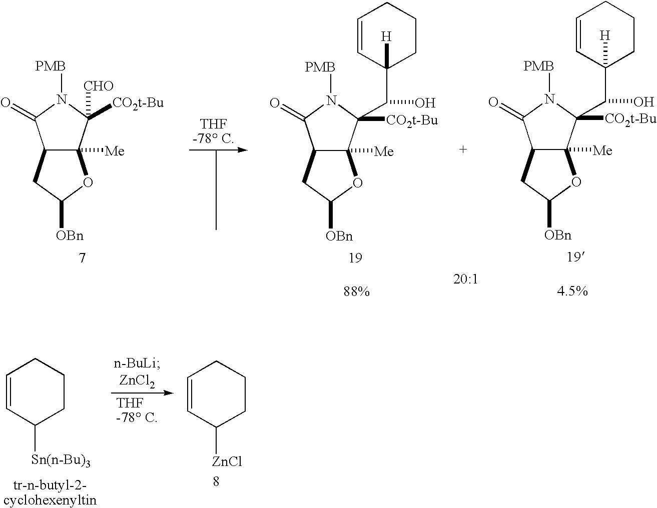

Cyclohexenyl adduct 19. A solution of tri-n-butyl-2-cyclohexenyltin (Miyake et al. Chem. Lett. 1992, 507-508; incorporated herein by reference) in (501 mg, 1.35 mmol) in THF (1.5 ml) was treated with n-BuLi (2.5M in hexanes, 515 μl, 1.29 mmol) at −78° C. After 30 min, the mixture was further treated with ZnCl₂ (1M in Et₂₀, 1.32 ml, 1.32 mmol). After 30 min, a solution of the aldehyde 7 (159 mg, 0.321 mmol) in THF (0.7 ml) was slowly added to the freshly prepared cyclohexenyl zinc reagent 8 and stirring was continued at −78° C. for 3 hours (Reddy et al. J. Am. Chem. Soc. 2004, 126, 6230; incorporated herein by reference). The reaction was quenched with saturated NH₄Cl solution, diluted with EtOAc, and sequentially washed with 1 M citric acid and saturated NaCl solution. The organic layer was concentrated in vacuo and the residue was purified by silica gel column chromatography (50% EtOAc in hexanes) to give the cyclohexenyl adduct 19 (pale yellow oil, 163 mg, 0.282 mmol, 88%) and its diastereomer 19′ (pale yellow oil, 8.3 mg, 0.018 mmol, 4.5%). Crystals of 19 and 19′ obtained from EtOH were used for confirmation of the structures by single-crystal X-ray analysis.

Data for the Major Adduct 19

¹H NMR (400 MHz, CDCl₃)

δ7.31 (d, 2H, J=8.7 Hz), 7.28-7.15 (m, 5H), 6.46 (d, 2H, J=8.7 Hz), 5.84 (d, 1H, J=9.7 Hz), 5.60 (d, 1H, J=9.7 Hz), 5.06 (d, 1H, J=5.6 Hz), 4.63 (d, 1H, J=15.1 Hz), 4.56 (d, 1H, J=12.6 Hz), 4.48 (d, 1H, J=15.1 Hz), 4.26 (d, 1H, J=12.6 Hz), 3.96 (m, 1H), 3.56 (s, 3H), 2.80 (d, 1H, J=8.5 Hz), 2.44 (d, 1H, J=13.6 Hz), 2.25-2.16 (m, 2H), 2.00-1.87 (m, 2H), 1.78-1.61 (m, 3H), 1.57-1.36 (m, 2H), 1.51 (s, 3H), 1.47 (s, 9H).

¹³C NMR (100 MHz, CDCl₃)

δ 177.44, 168.07, 158.41, 138.64, 132.42, 130.80, 129.10 (2C), 128.62 (2C), 128.07 (2C), 127.64, 126.00, 113.67 (2C), 102.45, 91.60, 83.07, 80.43, 76.86, 69.30, 55.53, 50.79, 48.34, 38.73, 35.41, 29.09, 28.47 (3C), 25.48, 21.96, 20.83.

FTIR (neat) νₘₐₓ: 3288, 2977, 2933, 2834, 1740, 1678, 1615, 1514, 1447, 1394, 1369, 1246, 1154, 1091, 1041, 1018, 992, 912, 841, 805, 733, 698.

HRMS (ESI) m/z calcd for C₃₄H₄₄NO₇ [M+H]⁺: 578.3118, found 578.3114.

[α]²³_{D}-34.1 (c 0.69, CHCl₃).

Data for the Minor Adduct 19′

¹H NMR (500 MHz, CDCl₃)

δ 7.28 (d, 2H, J=8.6 Hz), 7.27-7.14 (m, 5H), 6.54 (d, 2H, J=8.6 Hz), 5.68 (m, 1H), 5.60 (m, 1H), 5.04 (d, 1H, J=5.4 Hz), 4.74 (d, 1H, J=15.4 Hz), 4.63 (d, 1H, J=15.4 Hz), 4.62 (d, 1H, J=12.4 Hz), 4.24 (d, 1H, J=12.4 Hz), 3.93 (m, 1H), 3.61 (s, 3H), 2.84 (d, 1H, J=8.3 Hz), 2.46 (d, 1H, J=13.4 Hz), 2.18 (ddd, 1H, J=13.4, 8.3, 5.6 Hz), 1.97-1.79 (m, 3H), 1.61-1.13 (m, 5H), 1.50 (s, 3H), 1.47 (s, 9H).

¹³C NMR (125 MHz, CDCl₃)

δ 177.37, 167.76, 158.05, 138.27, 130.78, 130.68, 128.65, 128.19 (2C), 127.72 (2C), 127.19 (2C), 126.54, 113.35 (2C), 101.77, 91.66, 82.75, 79.72, 76.49, 68.87, 55.13, 50.36, 47.61, 39.14, 34.90, 28.02 (3C), 26.07, 25.29, 21.45, 19.72.

HRMS (ESI) m/z calcd for C₃₄H₄₄NO₇ [M+H]⁺: 578.3118, found 578.3120.

[α]²³_{D}-5.9 (c 1.7, CHCl₃)

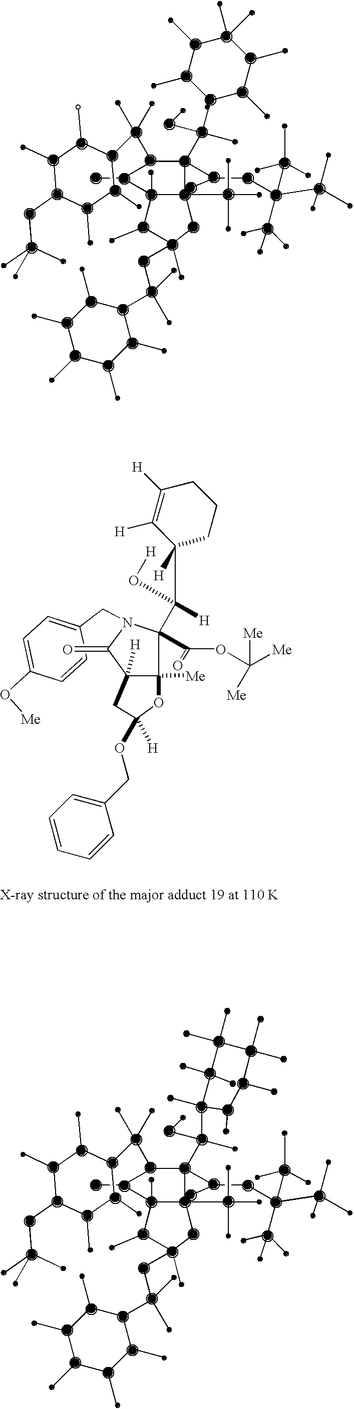

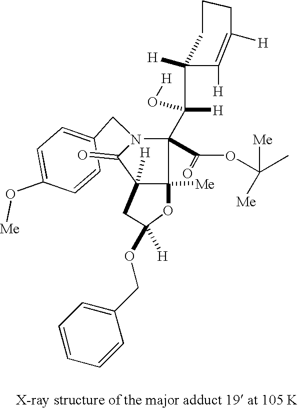

Appendix Synthesis of the Imidate Aldehyde and Reaction with Cyclohexenyl Zinc Chloride

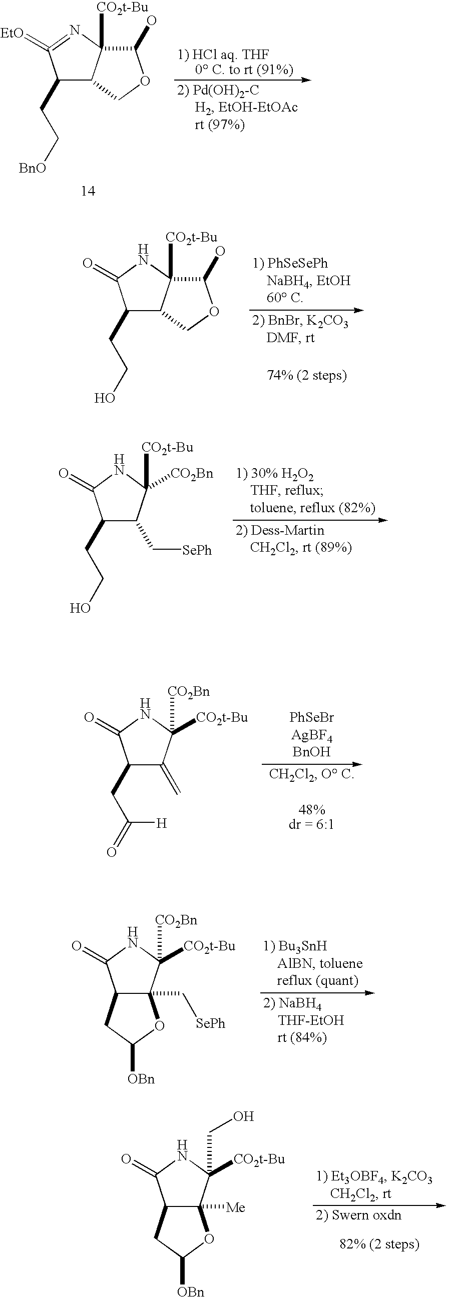

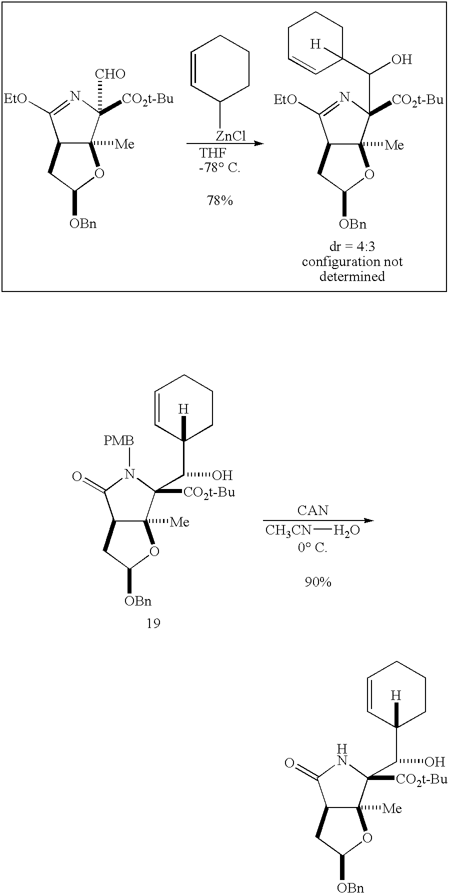

Lactam. A solution of ceric ammonium nitrate (CAN, 188 mg, 0.343 mmol) in H₂O (0.25 ml) was added to a solution of the cyclohexenyl adduct 19 (66.0 mg, 0.114 mmol) in CH₃CN (1.2 ml) at 0° C. After stirring for 6 h, the reaction was quenched with saturated Na₂SO₃ solution and partitioned between EtOAc and saturated NaHCO₃ solution. The organic layer was washed with saturated NaCl solution and concentrated in vacuo. The residue was purified by silica gel column chromatography (80% EtOAc in hexanes) to give the lactam (47.3 mg, 0.103 mmol, 90%) as a pale yellow oil.

¹H NMR (400 MHz, CDCl₃)

δ 7.15-7.35 (m, 5H), 6.00 (m, 1H), 5.81 (s, 1H), 5.52 (m, 1H), 5.01 (br, 1H), 4.58 (d, 1H, J=12.4 Hz), 4.37 (d, 1H, J=11.2 Hz), 3.92 (d, 1H, J=9.0 Hz), 2.74 d, 1H, J=7.9 Hz), 2.44 (d, 1H, J=13.2 Hz), 2.21 (m, 1H), 2.10 (m, 1H), 1.95 (m, 2H), 1.80-1.53 (m, 4H), 1.73 (d, 1H, J=9.8 Hz), 1.49 (s, 3H & s, 9H).

¹³C NMR (125 MHz, CDCl₃)

δ 177.41, 168.83, 138.43, 135.84, 128.63 (2C), 128.25, 127.67 (2C), 123.65, 101.63, 92.14, 82.96, 76.57, 75.79, 68.63, 51.02, 37.80, 35.05, 29.83, 28.41 (3C), 25.22, 21.40, 20.88.

FTIR (neat) νₘₐₓ: 3288, 2977, 2933, 2835, 1740, 1678, 1615, 1514, 1447, 1394, 1369, 1246, 1154, 1091, 1041, 1018, 992, 919, 841, 8.5, 733, 698.

HRMS (ESI) m/z calcd for C₂₆H₃₆NO₆ [M+H]⁺: 458.2543, found 458.2560.

[α]²³_{D}-64.0 (c 0.72, CHCl₃).

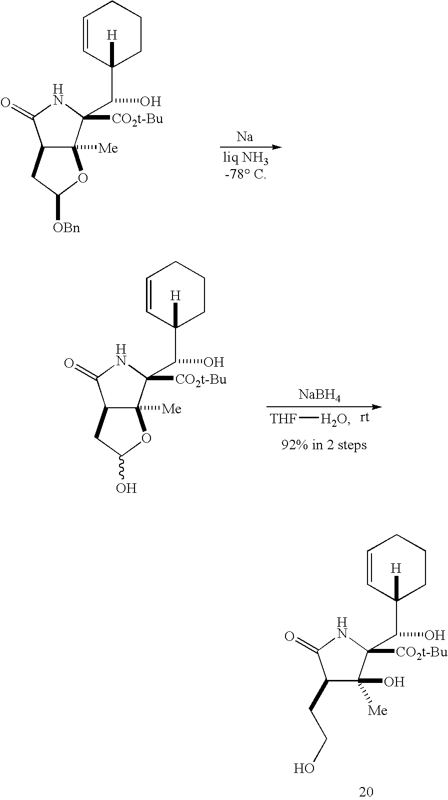

Triol 20. Sodium metal (Na, 81 mg, 3.5 mmol) was dissolved in liquid ammonia (8 ml) at −78° C. and the resultant dark blue mixture was stirred for 10 min. A solution of the benzyl glycoside (89.3 mg, 0.195 mmol) in THF (2 ml) was slowly added to the mixture and stirring was continued for 2 h. The reaction was quenched with NH₄Cl (solid, 300 mg) and dry ice-acetone bath was removed. All volatile materials were evaporated under N₂ stream and the white residue thus obtained was partitioned between EtOAc and saturated NaCl solution. The organic layer was further washed with saturated NaCl solution and concentrated in vacuo to give the crude hemiacetal (75.5 mg), which was used for the next reaction without purification.

A solution of the hemiacetal (75.5 mg) in THF—H₂O (2:1, 2.0 ml) was treated with NaBH₄ (22.9 mg, 0.61 mmol) at room temperature. After stirring for 30 min, the reaction mixture was diluted with EtOAc, sequentially washed with 1M citric acid and saturated NaCl solution, and concentrated in vacuo. The residue was purified by silica gel column chromatography (10% MeOH in CHCl₃) to give the triol 20 (70.0 mg, 0.189 mmol, 97% in 2 steps) as a white foam.

¹H NMR (500 MHz, CDCl₃)

δ 8.74 (brs, 1H), 6.07 (m, 1H), 5.79 (m, 1H), 5.28 (brs, 1H), 4.16 (d, 1H, J=8.4 Hz), 3.81 (m, 1H), 3.70 (m, 1H), 2.79 (dd, 1H, J=10.5, 2.1 Hz), 2.29 (m, 1H), 2.02 (m, 2H), 1.96 (m, 1H), 1.88-1.65 (m, 4H), 1.59 (m, 1H), 1.54 (s, 3H), 1.52 (s, 9H).

¹³C NMR (125 MHz, CDCl₃)

δ 180 67, 171 03, 135.48, 123.17, 84.16, 81.61, 79.82, 75.53, 62.37, 52.08, 38.32, 29.33, 28.02 (3C), 26.31, 24.80, 20.38, 19.80.

FTIR (neat) νₘₐₓ: 3306, 2977, 2930, 1713, 1684, 1669, 1371, 1288, 1253, 1156, 1046, 1019, 845, 700, 617.

HRMS (ESI) m/z calcd for C₁₉H₃₂NO₆ [M+H]⁺: 370.2230, found 370.2240.

[α]²³_{D}-53.3 (c 0.39, CHCl₃).

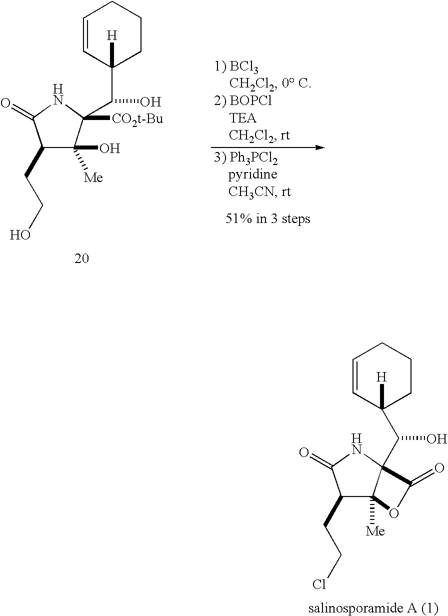

Salinosporamide A (1). A solution of the triol 20 (15.5 mg, 0.042 mmol) in CH₂Cl₂ (0.60 ml) was treated with BCl₃ (1M in CH₂Cl₂, 0.10 ml, 0.10 mmol) at 0° C. After 30 min, the reaction was quenched by addition of MeOH (50 μl) and the resultant mixture was concentrated to a small volume. The residue was dissolved in 5% EtOH in EtOAc and washed with saturated NaCl solution (×2). The organic layer was concentrated in vacuo and the crude carboxylic acid (13.7 mg, as a white film) was dissolved in a mixture of CH₂Cl₂ (0.50 ml) and triethylamine (TEA, 0.10 ml). After stirring for 10 min, BOPCl (23.4 mg, 0.092 mmol) was added at room temperature and stirring was continued for 16 h. The reaction mixture was diluted with EtOAc, sequentially washed with 1M citric acid solution and saturated NaCl solution, and passed through a small pad of silica gel (EtOAc 100%). The filtrate was concentrated in vacuo and the residue (7.7 mg, as a white film) was dissolved in a mixture of CH₃CN (0.20 ml) and pyridine (0.20 ml). Ph₃PCl₂ (16.9 mg, 0.051 mmol) was added to the mixture at room temperature and stirring was continued for 4 h. The reaction mixture was diluted with EtOAc, sequentially washed with saturated CuSO₄ solution and saturated NaCl solution, and concentrated in vacuo. The residue was purified by silica gel column chromatography (50% EtOAc in hexanes) to give salinosporamide A (1) (6.8 mg, 0.022 mmol, 51% in 3 steps) as a white film. Crystallization of synthetic 1 from EtOAc/cyclohexane gave colorless needles, which were used for further confirmation of the structure by single-crystal X-ray analysis.

¹H NMR (500 MHz, pyridine-d5)

δ 10.62 (s, 1H), 6.42 (d, 1H, J=10.0 Hz), 5.88 (m, 1H), 4.96 (brs, 1H), 4.26 (m, 1H), 4.13 (m, 1H), 4.02 (m, 1H), 3.18 (t, 1H, J=7.1 Hz), 2.85 (m, 1H), 2.49 (m, 1H), 2.36-2.29 (m, 2H), 2.07 (s, 3H), 1.91 (m, 2H), 1.65-1.72 (m, 2H), 1.37 (m, 1H).

¹³C NMR (125 MHz, pyridine-d5)

δ 176.93, 169.44, 129.09, 128.69, 86.32, 80.35, 70.99, 46.18, 43.29, 39.31, 29.01, 26.48, 25.36, 21.73, 20.00.

FTIR (neat) νₘₐₓ: 3389, 2926, 1826, 1702, 1432, 1385, 1226, 1080, 1022, 833, 778.

HRMS (ESI) m/z calcd for C₁₅H₂₁ClNO₄ [M+H]⁺: 314.1159, found 314.1174.

[α]²³_{D}-73.0 (c 0.40, MeOH), −73.2 (c 0.49, MeOH, in Reddy et al. J. Am. Chem. Soc. 126:6230, 2004; incorporated herein by reference), −72.9 (c 0.55, MeOH, in Feling et al. Angew. Chem. Int. Ed. 2003, 42, 355-357; incorporated herein by reference).

mp 166-167° C. (168-170° C. in Reddy et al. J. Am. Chem. Soc. 126:6230, 2004; incorporated herein by reference; 169-171° C. in Feling et al. Angew. Chem. Int. Ed. 2003, 42, 355-357; incorporated herein by reference).

X-Ray Structure of Salinosporamide A (1) (at 100K).

The unit cell contained four independent molecules A-D with different conformations.

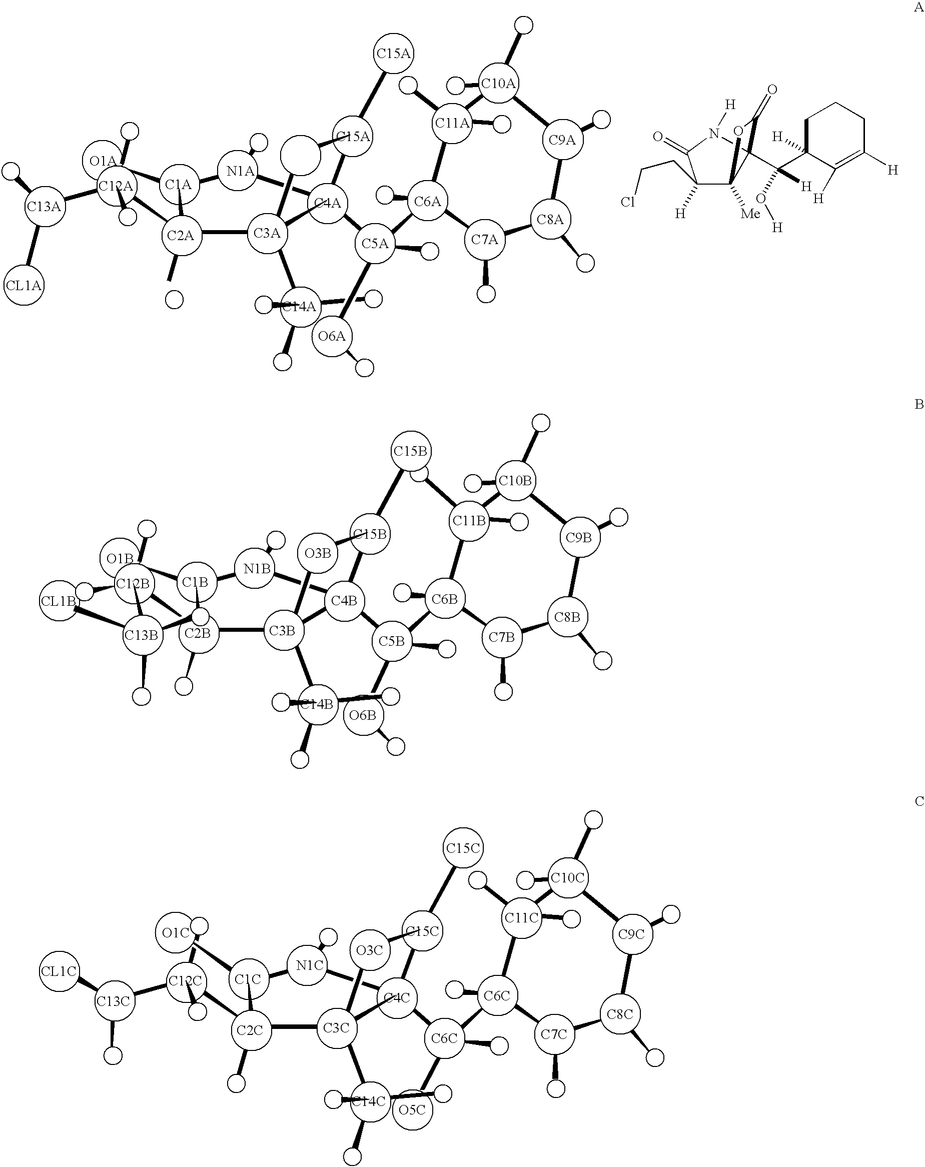

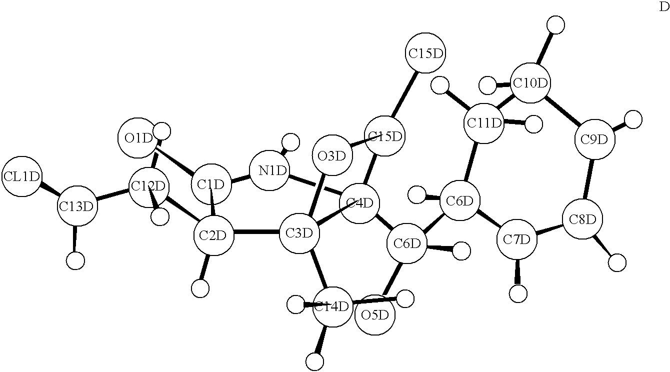

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method of preparing a compound of formula: 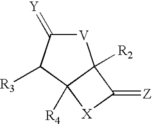
wherein Z is O;
Y is O, S, C(R₆), or NR₆, wherein each occurrence of R₆ is independently hydrogen or lower alkyl;
X is O, S, C(R₇)₂, or NR₇, wherein each occurrence of R₇ is independently hydrogen or lower alkyl;
V is NR₁, wherein each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is —CH(OH)R_{B}′, wherein R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}″; ═O; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is methyl; the method comprising steps of:
(a) providing a pyrroglutamate derivative of formula: 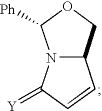
(b) reacting the pyrroglutamate derivative derivative with a vinyl nucleophile to generate a compound of formula: 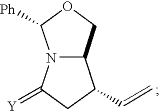
(c) alkylating the resulting compound at C-2 to yield a compound of formula: 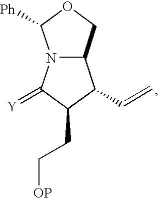
wherein P is an oxygen-protecting group;
(d) oxonolysing followed by reductive treatment of the carbon-carbon double bond to yield a primary alcohol of formula: 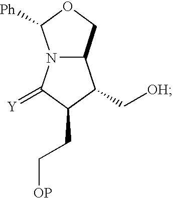
(e) reacting the primary alcohol with ClCO₂R, wherein R is C₁-C₆ alkyl, preferably ethyl, to yield an ethyl carbonate of formula: 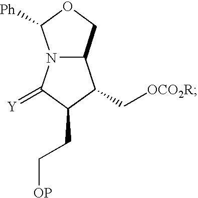
(f) removing the N,O-acetal protecting group by treatment with acid;
(g) oxidizing the resulting primary alcohol to yield the carboxylic acid of formula: 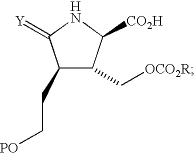
(h) esterifying the resulting carboxylic acid;
(i) treating the resulting ester with Meerwein reagent to yield a compound of formula: 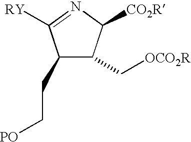
wherein R′ is C₁-C₆ alkyl, preferably t-butyl;
(j) treating the compound with a base to form the lactime ether of formula: 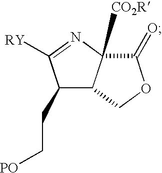
(k) treating the lactime ether with acid to afford the lactam;
(l) protecting the nitrogen of the lactam;
(m) removing the oxygen-protecting group to yield the compound of formula: 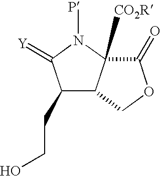
wherein P′ is a nitrogen protecting group, preferably PMB;
(n) opening the lactone ring with phenylselenium anion to yield a compound of formula: 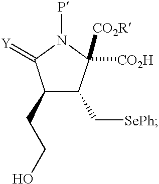
(o) protecting the carboxylic acid functional group;
(p) oxidizing the selenide and unprotected alcohol to yield a compound of formula: 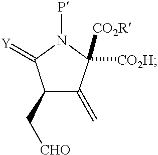
(q) cyclizing the compound to form an intermediate hemiacetal of formula: 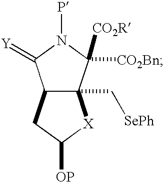
(r) deselenylating via a radical reaction to yield a compound of formula: 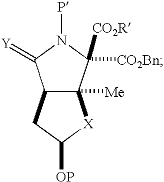
(s) reducing and oxidizing the benzyl ester to form the aldehyde of formula: 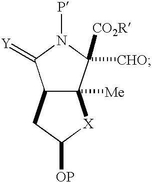
(t) treating the aldehyde with a nucleophile to yield a compound of formula: 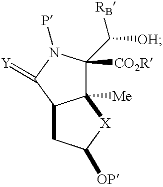
(u) removing the nitrogen-protecting group;
(v) opening the glycoside to yield the triol of formula: 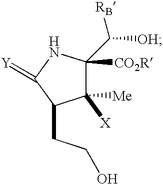
(w) hydrolyzing the alkylester;
(x) lactonizing under suitable conditions to form the 4-membered lactone; and
(y) derivatizing the primary alcohol to yield a compound of formula:

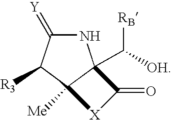

2. A method of preparing salinosporamide A and analogues thereof, the method comprising steps of:
(a) providing a pyrroglutamate derivative of formula: 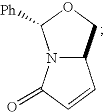
(b) reacting the pyrroglutamate derivative derivative with a vinyl nucleophile to generate a compound of formula: 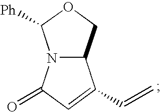
(c) alkylating the resulting compound at C-2 to yield a compound of formula: 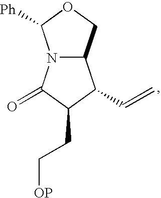
wherein P is an oxygen-protecting group;
(d) oxonolysing followed by reductive treatment of the carbon-carbon double bond to yield a primary alcohol of formula: 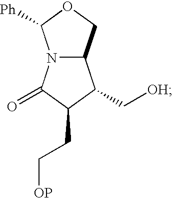
(e) reacting the primary alcohol with ClCO₂R, wherein R is C₁-C₆ alkyl, preferably ethyl, to yield an ethyl carbonate of formula: 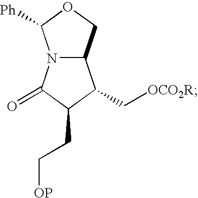
(f) removing the N,O-acetal protecting group by treatment with acid;
(g) oxidizing the resulting primary alcohol to yield the carboxylic acid of formula: 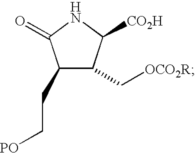
(h) esterifying the resulting carboxylic acid;
(i) treating the resulting ester with Meerwein reagent to yield a compound of formula: 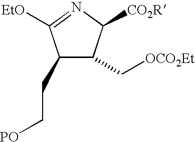
wherein R′ is C₁-C₆ alkyl, preferably t-butyl;
(j) treating the compound with a base to form the lactime ether of formula: 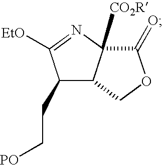
(k) treating the lactime ether with acid to afford the lactam;
(l) protecting the nitrogen of the lactam;
(m) removing the oxygen-protecting group to yield the compound of formula: 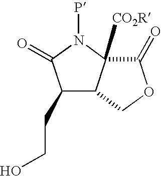
wherein P′ is a nitrogen protecting group, preferably PMB;
(n) opening the lactone ring with phenylselenium anion to yield a compound of formula: 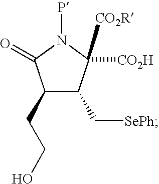
(o) protecting the carboxylic acid functional group;
(p) oxidizing the selenide and unprotected alcohol to yield a compound of formula: 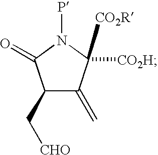
(q) cyclizing the compound to form an intermediate hemiacetal of formula: 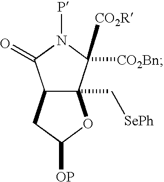
(r) deselenylating via a radical reaction to yield a compound of formula: 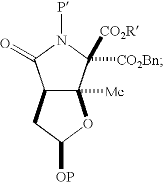
(s) reducing and oxidizing the benzyl ester to form the aldehyde of formula: 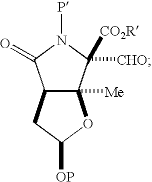
(t) treating the aldehyde with a cyclohexenyl zinc reagent to yield a compound of formula: 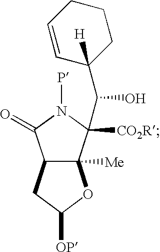
(u) removing the nitrogen-protecting group;
(v) opening the glycoside to yield the triol of formula: 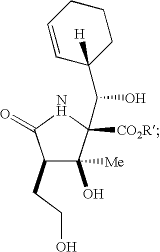
(w) hydrolyzing the alkylester;
(x) lactonizing under suitable conditions to form the 4-membered lactone; and
(y) chlorinating the primary alcohol to yield salinosporamide A:

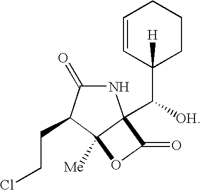

3. A method of preparing a compound of formula: 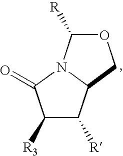
the method comprising steps of:
reacting a compound of formula: 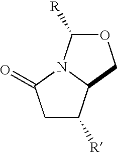
wherein R and R′ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl;
with a reagent of formula:
R₃—X
wherein R₃ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
X is halogen, —OTs, or other leaving group;
under suitable conditions to form a compound of formula: 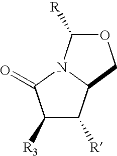
wherein R, R′, and R₃ are defined as above.

4. The method of claim 3, wherein R is phenyl

5. The method of claim 3, wherein R′ is C═CH₂.

6. The method of claim 3, wherein R₃ is CH₂CH₂R_{C}.

7. A method of preparing a compound of formula: 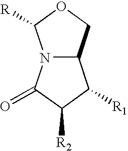
wherein R, R₁, and R₂ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl, the method comprising steps of:
reacting a pyrroglutamate derivative of formula: 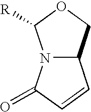
wherein R is defined above;
with a nucleophile under suitable condition in a 1,4-addition reaction; and
further reacting the 1,4-addition product under suitable conditions with an electrophile to yield a product of the formula: 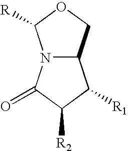
wherein R, R₁, and R₂ are defined as above.

8. The method of claim 7, wherein R is phenyl.

9. The method of claim 7, wherein R₁ is vinyl.

10. The method of claim 7, wherein R₂ is —CH₂CH₂OBn.

11. The method of claim 7, wherein the nucleophile is vinyl cuprate.

12. The method of claim 7, wherein the electrophile is an aliphatic iodide.

13. A method of preparing a compound of formula: 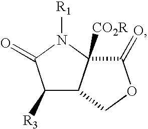
the method comprising steps of:
reacting a compound of formula: 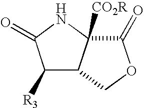
wherein R is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and
R₃ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
with a reagent of formula:
R₁—X
wherein R₁ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —OR_{A}; ═O; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
X is halogen, —OTs, or other leaving group.
under suitable conditions to form a compound of formula: 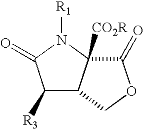
wherein R, R₁, and R₃ are defined as above.

14. The method of claim 13, wherein R is t-butyl.

15. The method of claim 13, wherein R₃ is CH₂CH₂R_{C}.

16. A method of preparing a compound of formula: 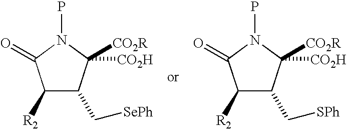
wherein P is hydrogen or a nitrogen-protecting group;
R and R₂ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; the method comprising step of:
reacting a lactone of formula: 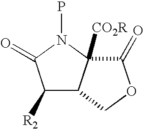
wherein R, R₂, and P are defined as above, with a phenylselenium anion or thiolate under suitable conditons to yield a compound of formula: 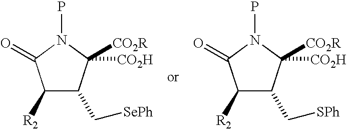
wherein R, R₂, and P are defined as above.

17. The method of claim 16, wherein P is PMB.

18. The method of claim 16, wherein R is t-butyl.

19. The method of claim 16, wherein R₂ is —CH₂CH₂OH.

20. A method of preparing a compound of formula: 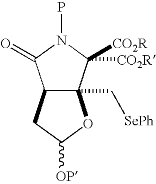
wherein P is hydrogen or a nitrogen-protecting group;
P′ is an oxygen-protecting group; and
R and R′ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; the method comprising steps of:
reacting a lactam of formula: 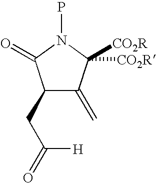
wherein P, R, and R′ are defined as above; with phenylseleneyl bromide and AgBF4 under suitable conditions to yield a cyclization product of formula:

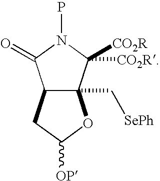

21. The method of claim 20, wherein P is PMB.

22. The method of claim 20, wherein P′ is benzyl.

23. The method of claim 20, wherein R is t-butyl.

24. The method of claim 20, wherein R′ is benzyl.

25. The method of claim 20, further compising the step of deselenylating the cyclization product.

26. The method of claim 25, wherein the step of deselenylating is accomplished using AIBN and n-BuSnH.

27. A method of preparing a compound of formula: 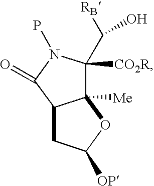
the method comprising steps of:
reacting a compound of formula: 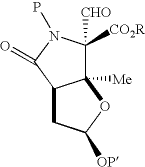
wherein R, P, and P′ are independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl;
with a nucleophile of formula:
^{⊖}R_{B}′
wherein R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —OR_{B}″; ═O; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
under suitable conditions to form a compound of formula: 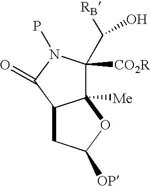
wherein R, P, P′, and R_{B}′ are defined as above.

28. The method of claim 27, wherein P is PMB.

29. The method of claim 27, wherein P′ is benzyl.

30. The method of claim 27, wherein R is t-butyl.

31. A compound of formula: 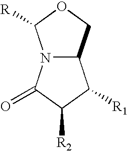
wherein R, R₁, and R₂ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl.

32. The compound of claim 31, wherein R is phenyl or substituted phenyl.

33. The compound of claim 31, wherein R is phenyl.

34. The compound of claim 31, wherein R₁ is vinyl.

35. The compound of claim 31, wherein R₂ is —CH₂CH₂OP, wherein P is hydrogen or an oxygen-protecting group.

36. The compound of claim 31, wherein P is benzyl.

37. A compound of formula: 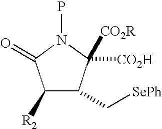
wherein P is hydrogen or a nitrogen-protecting group; and
R and R₂ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl.

38. The compound of claim 37, wherein P is PMB.

39. The compound of claim 37, wherein R is C₁-C₆ alkyl.

40. The compound of claim 37, wherein R is t-butyl.

41. The compound of claim 37, wherein R₂ is —CH₂CH₂OH.

42. A compound of formula: 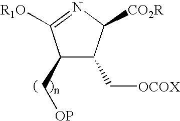
wherein R is hydrogen or C₁-C₆ alkyl;
R₁ is methyl, ethyl, or a silyl-protecting group;
n is 2, 3, or 4;
P is hydrogen or an oxygen-protecting group; and
X is hydrogen, halogen, alkoxy, alkylthioxy, alkylamino, or dialkylamino.

43. The compound of claim 42, wherein R is t-butyl.

44. The compound of claim 42, wherein R₁ is ethyl.

45. The compound of claim 42, wherein n is 2.

46. The compound of claim 42, wherein P is benzyl.

47. The compound of claim 42, wherein P is hydrogen.

48. The compound of claim 42, wherein X is —OMe.

49. The compound of claim 42, wherein X is —OEt.

50. The compound of claim 42, wherein X is halogen.

51. A compound of formula: 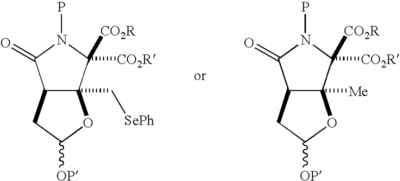
wherein P is hydrogen or a nitrogen-protecting group;
P′ is an oxygen-protecting group; and
R and R′ are independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl.

52. The compound of claim 51, wherein P is PMB.

53. The compound of claim 51, wherein P′ is benzyl.

54. The compound of claim 51, wherein R is t-butyl.

55. The compound of claim 51, wherein R′ is benzyl.

56. A compound of the formula: 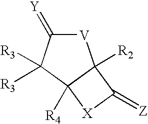
wherein Z is O, S, C(R₅), or NR₅, wherein each occurrence of R₅ is independently hydrogen or lower alkyl;
Y is O, S, C(R₆), or NR₆, wherein each occurrence of R₆ is independently hydrogen or lower alkyl;
X is O, S, C(R₇)₂, or NR₇, wherein each occurrence of R₇ is independently hydrogen or lower alkyl;
V is O, S, C(R₁)₂, or NR₁, wherein each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof, with the proviso that (1) when Y is oxygen, V is NH, X is oxygen, Z is oxygen, R₄ is methyl, R₃ is —CH₂CH₂Cl, then R₂ is not 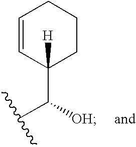
(2) when Y is oxygen, V is NH, X is oxygen, Z is oxygen, R₄ is hydrogen, R₃ is methyl, then R₂ is not

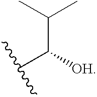

57. The compound of claim 56, wherein Z is oxygen.

58. The compound of claim 56, wherein Y is oxygen.

59. The compound of claim 56, wherein X is oxygen.

60. The compound of claim 56, wherein X is NH.

61. The compound of claim 56, wherein V is NR₁.

62. The compound of claim 56, wherein V is NH.

63. The compound of claim 56, wherein V is oxygen.

64. The compound of claim 56, wherein X is oxygen; Y is oxygen; Z is oxygen; and V is NR₁.

65. The compound of claim 56, wherein X is oxygen; Y is oxygen; Z is oxygen; and V is NH.

66. The compound of claim 56, wherein at least one R₃ is hydrogen.

67. The compound of claim 56 of formula:

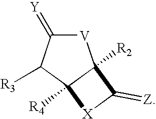

68. The compound of claim 56 of formula: 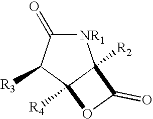
wherein X is O; Z is O; Y is O; and V is NR₁.

69. The compound of claim 68, wherein R₁ is hydrogen.

70. The compound of claim 56, wherein R₄ is alkyl.

71. The compound of claim 56, wherein R₄ is C₁-C₆ alkyl.

72. The compound of claim 56, wherein R₄ is methyl.

73. The compound of claim 56, wherein R₄ is hydrogen.

74. The compound of claim 56, wherein R₄ is not hydrogen or methyl.

75. The compound of claim 56 of formula: 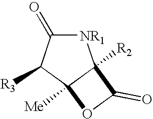
wherein X is O; Z is O; Y is O; V is NR₁; and R₄ is methyl.

76. The compound of claim 75, wherein R₁ is hydrogen.

77. The compound of claim 56, wherein R₁ is not hydrogen.

78. The compound of claim 56, wherein R₂ is

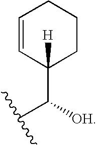

79. The compound of claim 56, wherein R₂ is not

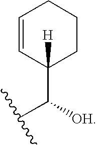

80. The compound of claim 56, wherein R₃ is —CH₂CH₂Cl.

81. The compound of claim 56, wherein R₃ is not —CH₂CH₂Cl.

82. The compound of claim 56, wherein R₃ is not

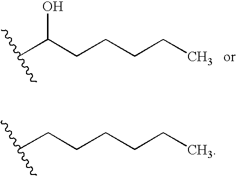

83. The compound of claim 56, wherein when X is oxygen, Y is oxygen, V is NH, Z is oxygen, R₂ is 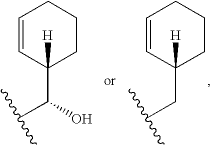
R₄ is methyl, then R₃ is not

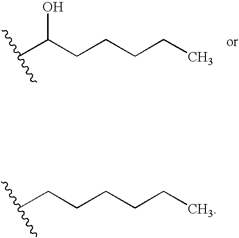

84. The compound of claim 56 of formula: 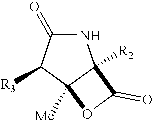
wherein X is O, Z is O; Y is O, V is NH; and R₄ is methyl.

85. The compound of claim 56 of the formula: 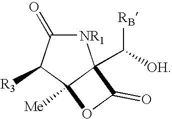
wherein R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}″; ═O; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

86. The compound of claim 56 of the formula: 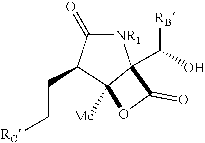
wherein R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}″; ═O; —C(═O)R_{C}″; —CO₂R_{C}″; —CN; —SCN; —SR_{C}″; —SOR_{C}″; —SO₂R_{C}″; —NO₂; —N(R_{C}″)₂; —NHC(O)R_{C}″; or —C(R_{C}″)₃; wherein each occurrence of R_{C}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and
R_{C}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}″; ═O; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

87. The compound of claim 56, wherein R₁ is hydrogen.

88. The compound of claim 56, wherein R₁ is substituted or unsubstituted, branched or unbranched, cyclic or acyclic aliphatic or heteroaliphatic.

89. The compound of claim 56, wherein R₁ is substituted or unsubstituted, branched or unbranched, cyclic or acyclic alkyl.

90. The compound of claim 56, wherein R₁ is C₁-C₆ alkyl.

91. The compound of claim 85 or 86, wherein R_{B}′ is a substituted or unsubstituted, branched or unbranched, cyclic or acyclic aliphatic or heteroaliphatic moiety.

92. The compound of claim 85 or 86, wherein R_{B}′ is a substituted or unsubstituted, branched or unbranched, cyclic aliphatic or heteroaliphatic moiety.

93. The compound of claim 85 or 86, wherein R_{B}′ is a substituted or unsubstituted, branched or unbranched cyclic aliphatic moiety.

94. The compound of claim 85 or 86, wherein R_{B}′ is a substituted or unsubstituted, branched or unbranched cyclic heteroaliphatic moiety.

95. The compound of claim 85 or 86, wherein R_{B}′ is a substituted or unsubstituted, branched or unbranched 5- or 6-membered carbocyclic ring.

96. The compound of claim 85 or 86, wherein R_{B}′ is a substituted or unsubstituted, branched or unbranched 5- or 6-membered heterocyclic ring.

97. The compound of claim 86, wherein R_{C}′ is a halogen.

98. The compound of claim 86, wherein R_{C}′ is chlorine.

99. The compound of claim 86, wherein R_{C}′ is hydroxyl, alkoxy, amino, alkylamino, dialkylamino, sulfhydryl, or acyl.

100. The compound of claim 85 or 86 of formula: 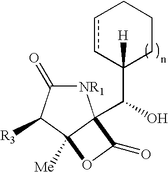
wherein n is 0, 1, or 2; and
the dashed line represents a bond or the absence of a bond.

101. The compound of claim 85 or 86, wherein R_{B}′ is substituted or unsubstituted, branched or unbranched aryl or heteroaryl.

102. The compound of claim 56 of formula:

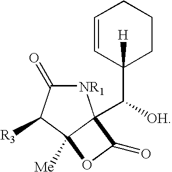

103. The compound of claim 56 of formula:

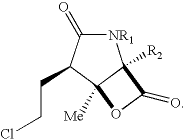

104. The compound of claim 56 of formula:

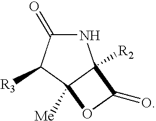

105. The compound of claim 56 of formula:

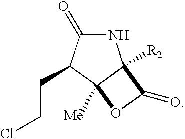

106. The compound of claim 56 of formula:

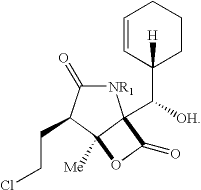

107. The compound of claim 56 of formula:

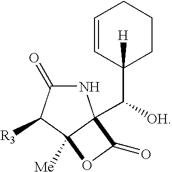

108. A compound of one of the formulae: 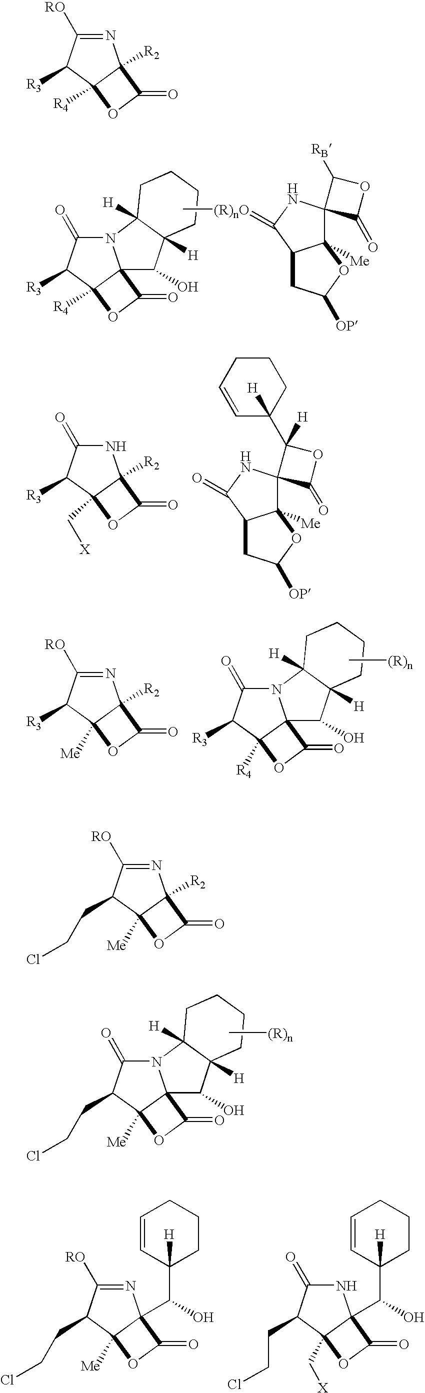
wherein R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; ═O; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R_{B}′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}″; ═O; —C(═O)R_{B}″; —CO₂R_{B}″; —CN; —SCN; —SR_{B}″; —SOR_{B}″; —SO₂R_{B}″; —NO₂; —N(R_{B}″)₂; —NHC(O)R_{B}″; or —C(R_{B}″)₃; wherein each occurrence of R_{B}″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
X is hydroxyl, alkoxy, halogen, sulfhydryl, alkylthioxy, amino, alkylamino, or dialkylamino;
P′ is selected from the group consisting of a hydrogen; cyclic or acyclic, substituted or unsubstituted, branched or unbranched aliphatic group; cyclic or acyclic, substituted or unsubstituted, branched or unbranched heteroaliphatic group; substituted or unsubstituted, branched or unbranched aryl group; substituted or unsubstituted, branched or unbranched heteroaryl group; cyclic or acyclic, substituted or unsubstituted, branched or unbranched acyl group; C₁-C₆ alkyl, and oxygen-protecteding groups; and
n is an integer between 0 and 8, inclusive; and pharmaceutically acceptable salts thereof.

109. A pharmaceutical composition comprising a compound of claim 56 and pharmaceutically acceptable excipient.

110. A method of treating a patient suffering from a proliferative disorder, the method comprising steps of:
administering to a patient suffering from a proliferative disorder a pharmaceutically effective amount of the compound of claim 56.

111. The method of claim 110, wherein the proliferative disorder is a cancer.

112. The method of claim 110, wherein the proliferative disorder is an autoimmune disease.

113. The method of claim 110, wherein the proliferative disorder is diabetic retinopathy.
